SEQUENCE LISTING (10 sequences)

SEQ ID NO: 1 - protein, 685 aa, Rattus norvegicus
MTPGSRSACRWALLLLAVLWPQRAAGSGIFQLRLQEFANERGMLANGRPCEPGCRTFFRICLKHYQATFSEGPCTFGNVSTPVLGTNSFVIRDKNSGSGRNPLQLPFNFTWPGTFSLNIQAWHTPGDDLRPETSPGNSLISQIIIQGSLAVGKNWKSDEQNNTLTRLRYSYRVVCSDNYYGDSCSRLCKKRDDHFGHYECQPDGSLSCLPGWTGKYCDQPICLSGCHEQNGYCSKPDECNCRPGWQGPLCNECIPHNGCRHGTCTIPWQCACDEGWGGLFCDQDLNYCTHHSPCKNGSTCSNSGPRGYTCTCLPGYTGEHCELELSKCASNPCRNGGSCKDHENSYHCLCPPGYYGQHCEHSTLTCADSPCFNGGSCRERNQGASYACECPPNFTGSNCEKKVDRCTSNPCANGGQCLNRGPSRTCRCRPGFTGTHCELHISDCARSPCAHGGTCHDLENGPVCTCPAGFSGRRCEVRITNDACASGPCFNGATCYTGLSPNNFVCNCPYGFVGSRCEFPVGLPPSFPWVAVSLGVGLVVLLVLLVMVAVAVRQLRLRRPDDDSREAMNNLSDFQKDNLIPAAQLKNTNQKKELEVDCGLDKSNCGKLQNHTLDYNLAPGFLGRGSTPGKYPHSDKSLGEKVPLRLHSEKPACRISAICSPRDSMYQSVCLISEERNECVIATEV

SEQ ID NO: 2 - protein, 257 aa, Rattus norvegicus
SGIFQLRLQEFANERGMLANGRPCEPGCRTFFRICLKHYQATFSEGPCTFGNVSTPVLGTNSFVIRDKNSGSGRNPLQLPFNFTWPGTFSLNIQAWHTPGDDLRPETSPGNSLISQIIIQGSLAVGKNWKSDEQNNTLTRLRYSYRVVCSDNYYGDSCSRLCKKRDDHFGHYECQPDGSLSCLPGWTGKYCDQPICLSGCHEQNGYCSKPDECNCRPGWQGPLCNECIPHNGCRHGTCTIPWQCACDEGWGGLFCDQ

SEQ ID NO: 3 - protein, 257 aa, Rattus norvegicus
SSIFQLRLQEFANERGMLANGRPCEPGCRTFFRICLKHYQATFSEGPCTFGNVSTPVLGTNSFVIRDKNSGSGRNPLQLPLNFTWPGTFSLIIQAWHTPGDDLRPETSPGNSLISQFIIQGSLAVGKNWKSDEQNNTLTRLRYSYRVVCSDNYYGDSCSRLCKKRDDYFGHYECQPDGSPSCLPGWTGEYCDQPICLSGCHEQNGYCSKPDECNCRPGWQGPLCNECIPHNGCRHGTCTIPWQCACDEGWGGLFCDQ

SEQ ID NO: 4 - protein, 1219 aa, Rattus norvegicus
MRSPRTRGRPGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTIQPDSIIEKASHSGMINPSRQWQTLKQNTGIAHFEYQIRVTCDDHYYGFGCNKFCRPRDDFFGHYACDQNGNKTCMEGWMGPECNKAICRQGCSPKHGSCKLPGDCRCQYGWQGLYCDKCIPHPGCVHGTCNEPWQCLCETNWGGQLCDKDLNYCGTHQPCLNRGTCSNTGPDKYQCSCPEGYSGPNCEIAEHACLSDPCHNRGSCKETSSGFECECSPGWTGPTCSTNIDDCSPNNCSHGGTCQDLVNGFKCVCPPQWTGKTCQLDANECEAKPCVNARSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSESGGKFTCDCNKGFTGTYCHENINDCEGNPCTNGGTCIDGVNSYKCICSDGWEGAHCENNINDCSQNPCHYGGTCRDLVNDFYCDCKNGWKGKTCHSRDSQCDEATCNNGGTCYDEVDTFKCMCPGGWEGTTCNIARNSSCLPNPCHNGGTCVVNGDSFTCVCKEGWEGPICTQNTNDCSPHPCYNSGTCVDGDNWYRCECAPGFAGPDCRININECQSSPCAFGATCVDEINGYQCICPPGHSGAKCHEVSGRSCITMGRVILDGAKWDDDCNTCQCLNGRVACSKVWCGPRPCQLHKGHGECPNGQSCIPVLDDQCFVRPCTGAGECRSSSLQPVKTKCTSDSYYQDNCANITFTFNKEMMSPGLTTEHICSELRNLNILKNVSAEYSIYIACEPSLSANNEIHVAISAEDIRDDGNPVKEITDKIIDLVSKRDGNSSLIAAVAEVRVQRRPLKNRTDFLVPLLSSVLTVAWVCCLVTAFYWCVRKRRRKPSSHTHSAPEDNTTNNVREQLNQIKNPIEKHGANTVPIKDYENKNSKMSKIRTHNSEVEEDDMDKHQQKVRFAKQPVYTLVDREEKAPSGTPTKHPNWTNKQDNRDLESAQSLNRMEYIV

SEQ ID NO: 5 - protein, 264 aa, Rattus norvegicus
SGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTIQPDSIIEKASHSGMINPSRQWQTLKQNTGIAHFEYQIRVTCDDHYYGFGCNKFCRPRDDFFGHYACDQNGNKTCMEGWMGPECNKAICRQGCSPKHGSCKLPGDCRCQYGWQGLYCDKCIPHPGCVHGTCNEPWQCLCETNWGGQLCDK

SEQ ID NO: 6 - protein, 317 aa, Homo sapiens
YFELQLSALRNVNGELLSGACCDGDGRTTRAGGCGHDECDTYVRVCLKEYQAKVTPTGPCSYGHGATPVLGGNSFYLPPAGAAGDRARARARAGGDQDPGLVVIPFQFAWPRSFTLIVEAWDWDNDTTPNEELLIERVSHAGMINPEDRWKSLHFSGHVAHLELQIRVRCDENYYSATCNKFCRPRNDFFGHYTCDQYGNKACMDGWMGKECKEAVCKQGCNLLHGGCTVPGECRCSYGWQGRFCDECVPYPGCVHGSCVEPWQCNCETNWGGLLCDKDLNYCGSHHPCTNGGTCINAEPDQYRCTCPDGYSGRNCE

SEQ ID NO: 7 - protein, 305 aa, Homo sapiens
SGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCE

SEQ ID NO: 8 - protein, 2555 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 9 - protein, 243 aa, Artificial Sequence
  Anti-CD33 single-chain variable fragment
SEVQLQQSGPEVVKPGASVKMSCKASGYTITDSNIHWVKQSRGKSLEWIGYIYPYNGGTDYNQKFKNKATLTVDNPSSTAYMELRSLTSEDSAVYYCVNGNPWLAYWGQGTLVTVSAGGGGSGGGGSGGGGSDIVLTQSPASLAVSLGQRATISCRASESLDNYGIRFLTWFQQRPGQPPKLLIYAASNQGSGVPARFSGSGSGTEFSLNIHPMEEDDTAIYFCQQTKEVPWSFGGGTKLEIK

SEQ ID NO: 10 - protein, 765 aa, Artificial Sequence
  Synthetic
SSIFQLRLQEFANERGMLANGRPCEPGCRTFFRICLKHYQATFSEGPCTFGNVSTPVLGTNSFVIRDKNSGSGRNPLQLPLNFTWPGTFSLIIQAWHTPGDDLRPETSPGNSLISQFIIQGSLAVGKNWKSDEQNNTLTRLRYSYRVVCSDNYYGDSCSRLCKKRDDYFGHYECQPDGSPSCLPGWTGEYCDQPICLSGCHEQNGYCSKPDECNCRPGWQGPLCNECIPHNGCRHGTCTIPWQCACDEGWGGLFCDQDLNYCTHHSPCKNGSTCSNSGPRGYTCTCLPGYTGEHCELELSKCASNPCRNGGSCKDHENSYHCLCPPGYYGQHCEHSTLTCADSPCFNGGSCRERNQGASYACECPPNFTGSNCEKKVDRCTSNPCANGGQCLNRGPSRTCRCRPGFTGTHCELHISDCARSPCAHGGTCHDLENGPVCTCPAGFSGRRCEVRITNDACASGPCFNGATCYTGLSPNNFVCNCPYGFVGSRCEGSGSGSGSGSEVQLQQSGPEVVKPGASVKMSCKASGYTITDSNIHWVKQSRGKSLEWIGYIYPYNGGTDYNQKFKNKATLTVDNPSSTAYMELRSLTSEDSAVYYCVNGNPWLAYWGQGTLVTVSAGGGGSGGGGSGGGGSDIVLTQSPASLAVSLGQRATISCRASESLDNYGIRFLTWFQQRPGQPPKLLIYAASNQGSGVPARFSGSGSGTEFSLNIHPMEEDDTAIYFCQQTKEVPWSFGGGTKLEIKEQKLISEEDLAAAHHHHHHHH

(19) United States
(12) Patent Application Publication
(10) Pub. No.: US 2022/0267437 A1
(43) Pub. Date: Aug. 25, 2022

(54) METHODS AND COMPOSITIONS FOR INDUCING NOTCH SIGNALING IN TUMOR MICROENVIRONMENTS

(71) Applicant: Fred Hutchinson Cancer Research Center, Seattle, WA (US)

(72) Inventors: Irwin D. Bernstein, Seattle, WA (US); Suzanne Furuyama, Seattle, WA (US)

(73) Assignee: Fred Hutchinson Cancer Research Center, Seattle, WA (US)

(21) Appl. No.: 17/631,399

(22) PCT Filed: Jul. 28, 2020
(86) PCT No.: PCT/US2020/043900

§ 371 Date: Jan. 28, 2022

Related U.S. Application Data
(60) Provisional application No. 63/001,136, filed on Mar. 27, 2020.
(60) Provisional application No. 62/880,014, filed on Jul. 29, 2019.

(51) Int. Cl. C07K 16/28; C07K 14/705

(52) U.S. Cl. CPC C07K 16/2803; C07K 14/705; C07K 2317/92; C07K 2317/55; C07K 2317/622; C07K 2317/75; C07K 2317/24

(57) ABSTRACT

The disclosure provides methods for inducing Notch signaling in a targeted manner within aggregations of cells. The methods include contacting the aggregation of cells with a bi-specific molecule that facilitates trans-binding of Notch receptor. The bi-specific molecule comprising a cell-targeting domain that specifically binds to a cell-specific antigen expressed in the aggregation of cells, and a Notch-binding domain that specifically binds to Notch receptor. In some aspects, the disclosed methods and reagents provide methods of promoting pro-inflammatory states in tumor micro-environments.

### CROSS-REFERENCE(S) TO RELATED APPLICATION(S)

This application claims the benefit of U.S. Provisional Application No. 62/880,014, filed Jul. 29, 2019, and U.S. Provisional Application No. 63/001,136, filed Mar. 27, 2020, the disclosures of which are incorporated herein by reference in their entireties.

### STATEMENT REGARDING SEQUENCE LISTING

The sequence listing associated with this application is provided in text format in lieu of a paper copy and is hereby incorporated by reference into the specification. The name of the text file containing the sequence listing is 72380_Sequence_final_2020-07-22.txt. The text file is 59 KB; was created on Jul. 22, 2020; and is being submitted via EFS-Web with the filing of the specification.

### BACKGROUND

The Notch signaling pathway is a highly conserved pathway that facilitates cell to cell signaling in metazoan animals. Mammalian Notch receptors (i.e. Notch1, 2, 3, and 4) are Type I transmembrane receptors that are initially expressed in precursor forms with an extracellular domain (NECD), a transmembrane domain, and an intracellular domain (NICD). The precursor is cleaved by a furin convertase to provide the mature receptor with two subunits. One subunit consists of the majority of the NECD, which remains noncovalently associated with the other subunit, which contains the transmembrane domain and NICD. The NECDs of the Notch receptors have a series of epidermal growth factor (EGF)-like repeats, which play a role in ligand interaction. After the EGF repeats (toward the C-terminus of the subunit) are three cysteine-rich LIN12 and Notch (LNR) repeats, which play a role in preventing ligand-independent signaling.

Signaling is typically initiated when the NECD binds to an appropriate ligand presented on the surface of an opposing cell. The canonical ligands, Jagged1(e.g., GenBank Accession No. AAC51731) Jagged2 (e.g., GenBank Accession No. AAD15562), Delta-like 1 (DLL1; e.g., GenBank Accession Nos. ABC26875 or NP005609), Delta-like 3 (DLL3; GenBank Accession Nos. NP_982353.1 or NP_058637.1), or Delta-like 4 (DLL4; e.g., GenBank Accession No.NP_061947.1) (the sequence of each accession number incorporated herein by reference), are also Type I transmembrane proteins and have an extracellular domain with an N-terminal region, a cysteine-rich Delta/Serrate/Lag2 (DSL) region, and a varying number of EGF repeats. The Notch signaling cascade is initiated by binding of a ligand to the Notch receptor on a neighboring cell. The ligand binding specifically results in a conformational change that exposes an S2 cleavage site in the NECD of the Notch receptor, permitting proteolysis. The conformational change is thought to result from a mechanical “tug” induced by the transendocytosis of the receptor-bound ligand into the ligand-expressing cell. Upon the cleavage of the Notch receptor at the S2 site, additional proteolysis occurs intracellularly to separate the NICD from the transmembrane domain. The active NICD then translocates to the nucleus and participates in a cascade of transcriptional activation and suppression pathways. Regulation of Notch signaling is mediated by several mechanisms. For example,

Notch receptors are subject to various post-translation modifications with the addition of sugars that can influence affinity for specific ligands or susceptibility to protease processing. Additionally, different Notch receptors have different affinities for the different ligands. Finally, cells expressing Notch receptors can also engage in cis-inhibition by co-expressing a ligand, typically distinct from the canonical ligands indicated above, that interacts with the Notch receptor without inducing proteolysis. The cis-binding of the Notch receptor prevents trans binding by a ligand expressed on a neighboring cell.

Because the general mechanism of Notch signaling operates with cell-to-cell contact, neighboring cells can mutually influence each other's gene transcription and subsequent development. These interactions permit lateral inhibition and, with the great diversity in potential regulatory mechanisms, allow groups of cells to organize and develop into complex tissues. Accordingly, Notch has been shown to play a key role in regulating cell proliferation, differentiation, development, and homeostasis. In adult mammals, Notch signaling plays a key role in numerous processes, including neural and hematopoietic stem cell renewal and differentiation, as well as the development of many immune cell subsets. For example, recent studies have suggested that Notch signaling mediates interactions of stem cells with cells within their specific microenvironments, also referred to as niches, contributing to stem cell quiescence. Notch signaling has also been implicated in the development and differentiation of immune cell subsets toward pro-inflammatory states.

For example, Notch signaling promotes differentiation of macrophages towards the M1 (i.e., pro-inflammatory) subset from a precursor or from an M2 (i.e., pro-tumor) subset. Notch signaling can also promote differentiation of monocytes into dendritic cells, which can interact with T cells to promote pro-inflammatory states.

Dysregulation of Notch signaling in different cell-types can result in a number of different inherited or acquired diseases, such as spondylocostal dysostoses, Alagille syndrome, Hajdu-Cheney syndrome, Alzheimer disease, cerebral autosomal dominant arteriopathy with subcortical infarcts, aortic valve disease, or leukoencephalopathy. Thus, Notch has been targeted for preventative and ameliorative therapies by modulating a variety of different targets regulating the Notch pathway. However, the utility of such an approach has heretofore been limited due to the fact that Notch plays a wide variety of critical roles throughout the body and that indirect modulation of normal Notch signaling in healthy tissues may lead to unacceptable toxicities and side-effects. This concept is illustrated by the observation that elevated Notch signaling is a tumor promoter of certain cancers, such as described above, but normal Notch signaling has also been found to function as a tumor suppressor in other cancers, including in some keratinocyte, pancreatic and hepatocellular carcinomas, and small-cell lung cancers. Thus, systemic or non-specific targeting of Notch signaling for one purpose can have deleterious effects throughout other cells and tissues in the body, reducing the utility of such treatments.

Accordingly, notwithstanding the advances in influencing Notch signaling, there remains a need for compositions and methods to selectively target cells for Notch modulation to while minimizing off-target effects. The present disclosure addresses this and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of inducing Notch signaling in an aggregation of cells comprising a first cell-type that expresses a cell-specific antigen and a second cell-type that expresses Notch. The method comprises contacting the aggregation of cells with a bi-specific molecule comprising a cell-targeting domain that specifically binds to the cell-specific antigen and a Notch-binding domain that specifically binds to Notch. Binding of the bi-specific molecule to the cell-specific antigen on a first cell of the first cell-type and trans-binding to Notch on a second cell of the second cell-type causes

Notch signaling in the second cell. In some embodiments, the first cell-type that expresses the cell-specific antigen and the second cell-type that expresses Notch are different cell-types. The aggregation of cells can be in a tumor microenvironment. In some embodiments, the first cell-type comprises tumor cells and the second cell-type comprises non-tumor cells in the tumor microenvironment, wherein binding of the bi-specific molecule to the cell-specific antigen on a tumor cell (i.e., the “first cell”) and trans-binding to Notch on a non-tumor cell (i.e., the “second cell”) causes Notch signaling in the non-tumor cell. The non-tumor cells comprise, stromal cells, endothelial cells, and immune cells, alone or in any combination.

In another aspect, the method provides a method of promoting a pro-inflammatory state in a tumor microenvironment comprising a tumor cell and a non-tumor cell. The method comprises administering to the tumor microenvironment a bi-specific molecule that comprises a cell targeting domain that specifically binds to a cell-specific antigen expressed by the tumor cell and a Notch binding domain that trans-binds to Notch expressed by a non-tumor cell in the tumor micro-environment, thereby inducing Notch signaling in the non-tumor cell.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 schematically illustrates the role of Notch signaling in inducing quiescence in cancer stem cells (CSCs).

FIG. 2 schematically illustrates the difference between trans-binding of Notch ligand from a neighboring cell (left panel) and cis-binding of a Notch ligand to the Notch receptor expressed on the same cell (middle panel). Trans-binding results in Notch activation, whereas cis-binding results in Notch inhibition. Inhibition via cis-binding of Notch is exploited by the administration of a bi-specific protein reagent (right panel) that binds both Notch and a cell-specific marker on the same cell. This mimics cis-binding inhibition for target cells of choice. A schematic design of an exemplary bi-specific protein reagent (BSP) is shown that combines the Notch ligand Delta4 with an affinity reagent that binds to the cell surface marker CD33. See also WO 2018/017827, incorporated herein by reference in its entirety.

FIG. 3 schematically illustrates an assay demonstrating the cis-inhibition induced by exposure to the BSP illustrated in FIG. 2 and described in more detail in WO 2018/017827, incorporated herein by reference in its entirety.

FIGS. 4A and 4B illustrate that the administration of the BSP illustrated in FIG. 2 surprisingly led to increased Notch signaling in CD33⁺ cells in vivo, as determined by monitoring expression of YFP, a Notch activation reporter, using an IVIS in vivo tumor imaging system (Perkin Elmer). FIG. 4A is a cartoon diagram of the subject mice receiving BSP administration. FIG. 4B shows representative images of BSP-treated and control mice with overlaid with detected YFP expression indicating Notch signaling.

FIGS. 5A and 5B illustrate the Notch expression in tumors pre-injection and day 2 post injection of the BSP. FIG. 5A shows representative mice pre and post control or BSP injection with an overlay of detected YFP expression in the tumors, indicating Notch signaling. FIG. 5B graphically illustrates the % change in YFP, a measure of Notch activation, relative to pre-injection of the BSP. Administration of the BSP led to a significant increase in Notch signaling in the CD33⁺ tumors in vivo.

FIG. 6 schematically illustrates a model of Notch receptor-ligand interactions in cell aggregations such as in tumor micro-environments. As illustrated, cells with Notch bound to BSP presented in trans are induced for Notch signaling, whereas cells with Notch bound to BSP presented in cis are inhibited for Notch signaling. The trans-binding is facilitated by the relative density and mutual proximity of neighboring cells that express the Notch receptor and cell-specific antigen (e.g., CD33 as illustrated).

FIGS. 7A and 7B illustrate an in vivo assay to assess the effect of BSP on Notch activation in mixed CD33⁺ and CD33⁻ tumors in mice. FIG. 7A is a cartoon diagram of the subject mouse with CD33⁺, CD33⁻, and mixed CD33⁺ & CD33⁻ tumors.

FIG. 7B shows representative images of BSP or control-treated mice with an overlay of detected YFP expression indicating Notch signaling. The mixed tumors were used to demonstrate that the BSP results in strong Notch signaling in the presence of mixed solid tumor setting.

FIGS. 8A and 8B illustrate the results of the mixed tumor assays illustrated in FIGS. 7A and 7B. FIG. 8A is a cartoon diagram of the subject mouse with CD33⁺, CD33⁻, and mixed CD33⁺ & CD33⁻ tumors. FIG. 8B graphically illustrates the % change in YFP expression relative to pre-injection levels, which indicate Notch signaling. The mixed CD33⁺ and CD33⁻ solid tumors exhibited a significant increase in Notch signaling as compared to the homogenous tumor types.

FIG. 9 is a schematic design of a modified bispecific protein reagent that favors cis-binding to enhance the inhibition effect.

FIG. 10 is a cartoon schematic illustrating the role of Notch in a heterogeneous tumor microenvironment, which typically present immuno-suppressive microenvironments that limit many immunotherapeutic strategies. An increase in Notch signaling induces the non-tumor cells in the tumor microenvironment towards a more pro-inflammatory state.

FIG. 11 is a cartoon schematic illustrating use of an exemplary bi-specific protein reagent to induce trans-binding to Notch in a heterogeneous tumor microenvironment. The trans-binding to Notch induces Notch signaling, which leads the non-tumor cells in the tumor microenvironment towards a more pro-inflammatory state and represents a strategy to overcome the challenge presented by the immuno-suppressive tumor micro environments.

FIGS. 12A-12C illustrates the design (FIG. 12A) and result (FIGS. 12B and 12C) of an assay to use the BSP to induce Notch signaling in a CD33+ 4T1 tumor microenvironment followed by characterization of the immune-phenotype of the tumor infiltrate as well as gene expression within isolated tumor-associated macrophages. Administration of the BSP altered immunophenotype and gene expression of tumor-associated myeloid cells.

FIGS. 13A and 13B graphically illustrate that bi-specific targeting to melanoma or breast tumors increases the percent of MHCII-expressing TAMs. 10⁶ Yummer1.7-CD33 melanoma cells (13A) or 10⁵ 4T1-CD33 cells (13B) were injected into the flank of C57 mice. At days 7, 9 and 12 post-Yummer1.7-CD33 cell injection and at day 5 post-4T1-CD33 cell injection, mice were intravenously injected with 3 mgs of bi-specific reagent or Hepes Buffered Saline as a control. At day 14 or 7 post-cell injection, melanoma or breast tumors, respectively were individually resected, minced with scissors/forceps and subjected to enzymatic digestion using the Tumor Dissociation Kit (Miltenyi). Cells were passed through a 100um strainer and stained with antibodies for flow cytometry. Cells were analyzed for immune-phenotype using FACS. Melanoma TAMs (CD45⁺ Lin^{lo} CD11b⁺ F4/80^{hi} CD169⁺ Ly6c⁻) or breast cancer TAMs (CD45⁺ Lin^{lo} CD11b⁺ F4/80^{hi} Ly6c⁻) were analyzed for MHCII expression.

FIG. 14 graphically illustrates reduced Yummer cell melanoma tumor growth following treatment with of the BSP reagent. 10⁶ Yummer1.7-CD33 melanoma cells were injected into the flank of C57 mice. At days 7, 9 and 12 post-cell injection, mice were intravenously injected with 3 mgs of bi-specific reagent or Hepes Buffered Saline as a control. At 14 days post-cell injection (experiment endpoint), reduced tumor growth was observed in mice treated with bi-specific in three independent experiments, unpaired t-test p-value=0.0433.

FIG. 15 is a series of photomicrographs demonstrating that bi-specific treatment increases macrophages that express MHCII within the tumor in a murine melanoma model. 10⁶ Yummer1.7-CD33 melanoma cells were injected into the flank of C57 mice. At days 7, 9 and 12 post-cell injection, mice were intravenously injected with 3 mgs of bi-specific reagent (BSP) or Hepes Buffered Saline (Control). At day 14 post-cell injection, tumors were resected, fixed in formalin, embedded in paraffin wax and cut into sections several microns thick. Sections were simultaneously stained with antibodies to F4/80 and MHCII and antibody binding measured using chromogenic detection. Digital images of stained slides were acquired using an Aperio ScanScope FL and analysis performed using Halo image analysis software. Number represents percent of F4/80/MHCII double positive cells among all F4/80 cells.

FIG. 16 is a series of photomicrographs demonstrating that bi-specific treatment increases macrophages that express iNOS within the tumor in a murine melanoma model. 10⁶Yummer1.7-CD33 melanoma cells were injected into the flank of 57 mice. At days 7, 9 and 12 post-cell injection, mice were intravenously injected with 3 mgs of bi-specific reagent (BSP) or Hepes Buffered Saline (Control). At day 14 post-cell injection, tumors were resected, fixed in formalin, embedded in paraffin wax and cut into sections several microns thick. Sections were stained with antibody to iNOS and antibody binding measured using chromogenic detection. Digital images of stained slides were acquired using an Aperio ScanScope FL and analysis performed using Halo image analysis software.

### DETAILED DESCRIPTION

As described above, there has been extensive development of therapeutic approaches to alter Notch signaling for treating various diseases, including cancer. However, considering the numerous and variable roles of Notch signaling in different tissues and cells throughout the body, interventions that broadly alter Notch signaling in multiple tissues can lead to toxicities and other adverse side-effects that limit their usage.

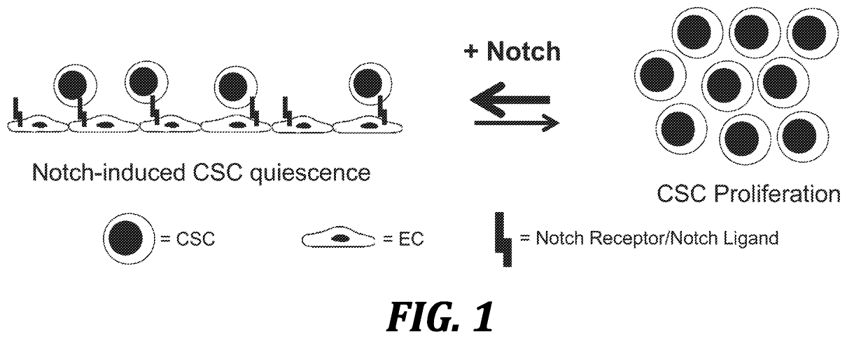

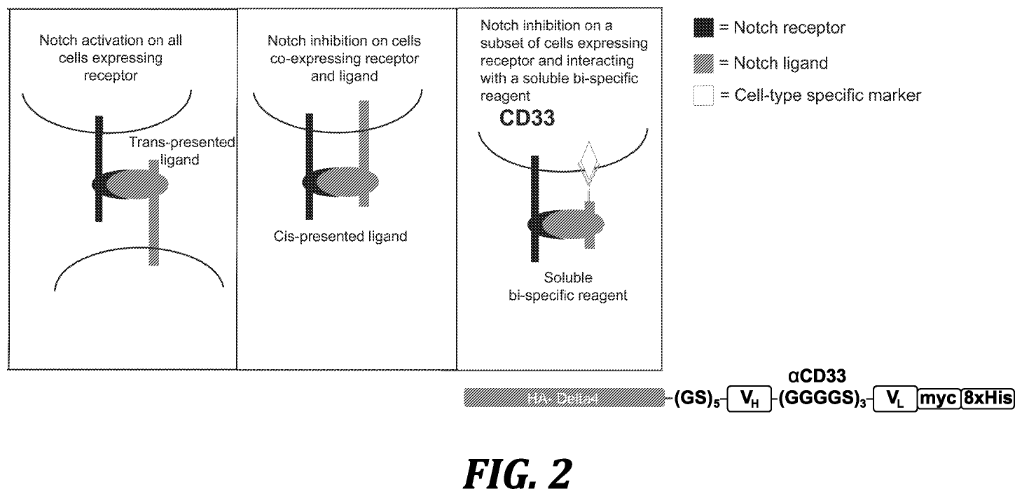

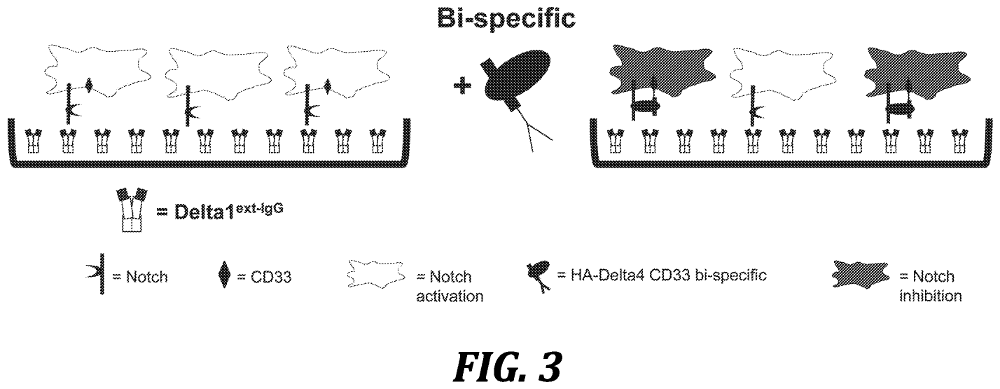

WO 2018/017827, incorporated herein by reference in its entirety, describes that Notch signaling induced by trans-binding of Notch ligand from neighboring cells induces CSC quiescence and longevity in cancer stem cells. See FIG. 1. Such signaling is detrimental to therapeutic interventions because the quiescent stem cells could remain in the patient and permit recurrence of cancer. Thus, to specifically inhibit this Notch stimulation, WO 2018/017827 describes the development of a bi-specific protein reagent (also referred to herein as “BSP” or a bi-specific reagent), that causes targeted Notch inhibition in the target cells by cis-binding the Notch receptor and a surface antigen specifically expressed on the same cell. The cis-binding mimicked naturally occurring interaction of Notch with ligand in cis, which prevents signaling from cognate ligands on adjacent cells that would otherwise induce Notch signaling. See FIG. 2. FIG. 3 illustrates a specific assay that demonstrated that cells bound by bi-specific reagent in cis were prevented from Notch stimulation in vitro.

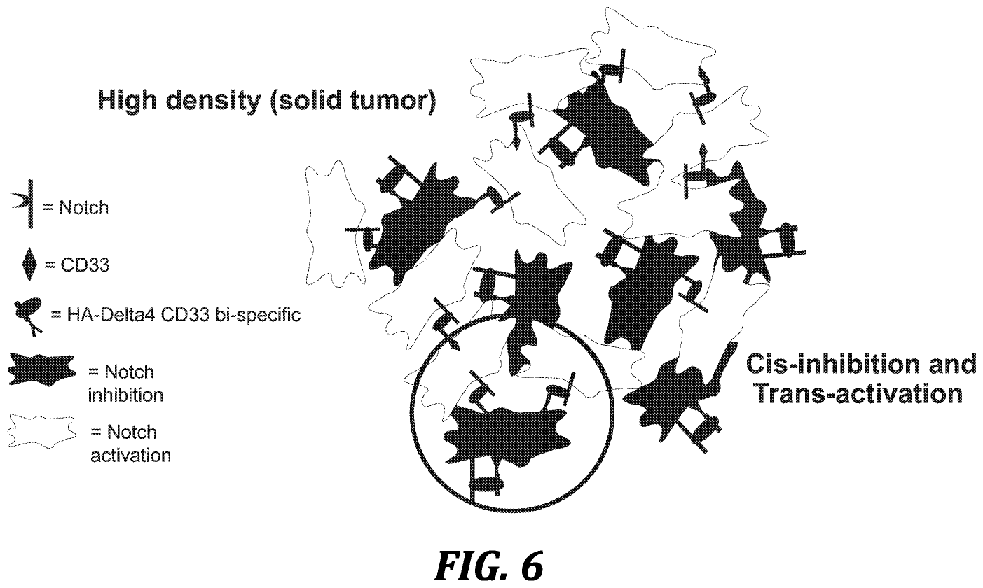

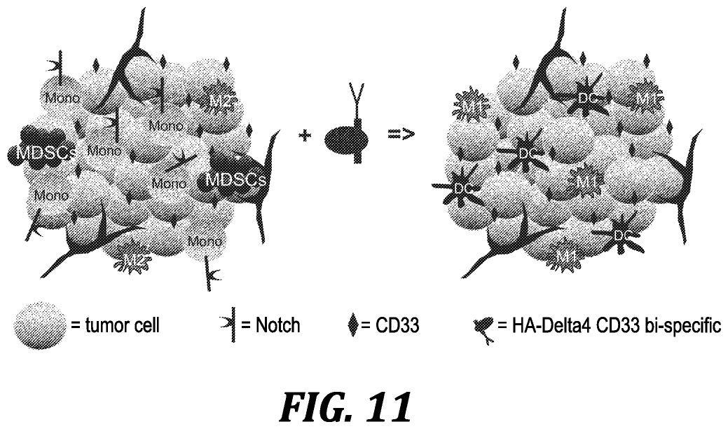

As described in more detail below, the inventors conducted further investigations of the bi-specific reagents disclosed in WO 2018/017827 and surprisingly discovered the opposite effect in the context of tumor microenvironments when administered in vivo. Without being limited to any particular theory, the inventors propose that the surprising induction of Notch stimulation instead of inhibition is due to the close aggregation of cells in a tumor microenvironment, where the bi-specific reagent specifically binds to a cell specific marker on one cell and the Notch receptor of a neighboring cell. See FIG. 6. This results in trans-binding of Notch receptor, i.e., where the bispecific reagent is also bound to an antigen on a different cell instead of the same cell. The trans-binding mimics the natural trans-binding of Notch receptor by it cognate ligand expressed by neighboring cells. This result presents a surprising and novel utility for the bi-specific reagent, namely the specific targeting of cells (e.g., non-tumor cells) in the tumor microenvironment for increased Notch signaling. The specific targeting is conferred by use of a marker specific for (e.g., substantially unique to) the tumor cells or other non-tumor cells or substrates (e.g., collagen) with increased presence in the tumor microenvironment. By specifically binding to the tumor cells or other elements within the tumor microenvironment, the bi-specific reagent can execute trans-binding of Notch receptor on a neighboring non-tumor cell, thereby stimulating Notch signaling. For many non-tumor cells in the tumor microenvironment, such as stromal cells, endothelial cells, and immune cells, this can promote a pro-inflammatory state, which can overcome or counteract the immunosuppressive conditions typical in many tumor microenvironments. See FIGS. 10 and 11. Such an approach can be used as a standalone therapy or in combination with other cancer therapeutic regimens, such as immune checkpoint inhibitors adoptive cell therapies (e.g., CAR T and CAR NK cell therapies, cancer-targeting antibodies), and the like.

In accordance with the foregoing, in one aspect, the disclosure provides a method of inducing Notch signaling in an aggregation of cells comprising a first cell-type that expresses a cell-specific antigen and a second cell-type that expresses Notch. The method comprises contacting the aggregation of cells with a bi-specific molecule comprising a cell-targeting domain that specifically binds to the cell-specific antigen and a Notch-binding domain that specifically binds to Notch receptor. Binding of the bi-specific molecule to the cell-specific antigen on a first cell of the first cell-type and trans-binding to Notch on a second cell of the second cell-type causes Notch signaling in the second cell.

In some embodiments, the first cell-type that expresses the cell-specific antigen and the second cell-type that expresses Notch are the same cell-type. These embodiments encompass scenarios where the cells are cancer or tumor cells wherein Notch signaling is anti-oncogenic. Thus, contacting the aggregation of cells with the bi-specific molecule would result in trans-binding of the Notch receptor of one tumor cell by the bi-specific molecule, which is also bound to a tumor specific antigen on a neighboring tumor cell of the same type. The trans-binding of the Notch receptor induces Notch signaling in the tumor cell, providing anti-oncogenic effects.

In other embodiments, the first cell-type that expresses the cell-specific antigen and the second cell-type that expresses Notch are different cell-types. Thus, the aggregation of cells is specifically targeted by virtue of the first cell-type expressing a substantially unique antigen. The bi-specific molecule binds to the cell-specific (e.g., substantially unique) antigen on a first cell of the first cell-type to facilitate trans-binding to Notch on a second cell of the second cell-type. The trans-binding of Notch on the second cell induces Notch signaling in the second cell.

In some embodiments, the aggregation of cells is in a tumor microenvironment. For example, the first cell-type can comprise tumor cells in the tumor microenvironment and the second cell-type can comprise non-tumor cells in the tumor microenvironment. In other embodiments, the first cell-type is a non-tumor cell that is present in the tumor microenvironment at higher levels compared to non-tumor environments. Binding of the bi-specific molecule to the cell-specific antigen (e.g., a tumor antigen or other antigen on a non-tumor cell that is predominant in the tumor microenvironment) on the first cell and trans-binding to Notch on a non-tumor cell (i.e., the “second cell”) causes Notch signaling in the non-tumor cell. The non-tumor cells can comprise stromal cells, endothelial cells, and/or immune cells, alone or in any combination. Thus, the second cell with trans-binding induction of Notch signaling can be a stromal cell, endothelial cell, immune cell, etc., and is specifically targeted for such induction of Notch signaling by virtue of being in close proximity to a tumor cell expressing a substantially unique antigen within a tumor microenvironment. The induction of Notch signaling using such a bi-specific molecule that requires specificity for tumor specific antigens creates a targeted induction of Notch within the tumor microenvironment while avoiding or reducing the likelihood of systemic or off target induction of Notch signaling.

The discussion presented here is generally in terms of targeting a “cell-specific antigen on a first cell of the first cell-type”. However, it is noted that the disclosure also encompasses embodiments where the antigen targeted by the cell-targeting domain is not necessarily an antigen on the cell, but is an extracellular substrate that is, e.g., produced by a first cell-type, and may be more prominent within the microenvironment defined by the aggregation of cells. An exemplary extracellular substrate includes, e.g., collagen, which can often be found at increased levels in a tumor micro-environment.

In some embodiments, the first cell-type comprises tumor cells and the second cell-type comprises immune cells. In other embodiments, the first cell-type comprises cells present in a tumor microenvironment and the second cell-type specifically comprises immune cells. Binding of the bi-specific molecule to the cell-specific antigen on a tumor cell (i.e., the “first cell”) and trans-binding to Notch on an immune cell (i.e., the “second cell”) causes Notch signaling in the immune cell. The Notch signaling can produce or promote an immune-responsive state in the tumor microenvironment. For example, the induction of Notch signaling in the immune cell by the trans-binding to Notch receptor on the immune cell in the tumor microenvironment promotes a pro-inflammatory phenotype in the immune cell.

In some embodiments, the immune cell (i.e., the “second cell”) is a monocyte and trans-binding of the bi-specific molecule to Notch on the monocyte promotes differentiation of the monocyte into a dendritic cell. Dendritic cells are professional antigen presentation cells that can interact with TH cells to stimulate an immune (e.g., an anti-tumor, pro-inflammatory response) response within the tumor micro-environment. In other embodiments, trans-binding of the bi-specific molecule to Notch on the immune cell promotes differentiation of macrophages from a pro-tumor phenotype (e.g., M2 subset of macrophages) towards an anti-tumor, pro-inflammatory phenotype (e.g., M1 subset of macrophages). The immune cell can be, for example, an M2 macrophage that upon trans-binding of the bi-specific molecule to Notch is induced to differentiate into an M1 macrophage. Alternatively, the immune cell is a macrophage precursor that is induced upon Notch signaling to differentiate into an M1 macrophage. In yet other embodiments, trans-binding of the bi-specific molecule to Notch on the immune cell promotes conversion of myeloid derived suppressor cells from an anti-inflammatory state to a pro-inflammatory state. Myeloid derived suppressor cells (MDSCs) are a heterogeneous group of immune cells originating from bone marrow stem cells and are characterized by a typically strong immunosuppressive activity. Typically, the infiltration of MDSCs into tumors is associated with poor treatment outcomes because they contribute to an immunosuppressive tumor microenvironment. However, upon induction of Notch signaling in the immune cells of the tumor microenvironment, the immunosuppressive function of the MDSCs is inhibited, thus rendering the overall microenvironment less immunosuppressive and more susceptible to immune responses and related therapies. These exemplary embodiments of immune-activation (e.g., pro-inflammatory response) are illustrated in FIGS. 10 and 11. In some embodiments, the trans-binding of the bi-specific molecule to Notch on an immune cells results in development of an anti-tumor phenotype on T cells, such as CD4⁺ and/or CD8+ T cells. In other embodiments, the trans-binding of the bi-specific molecule to Notch on an immune cells results in development of an anti-tumor phenotype on NK cells.

In another aspect, the disclosure provides a method of promoting a pro-inflammatory state in a tumor microenvironment comprising a tumor cell and a non-tumor cell. The method comprises administering to the tumor microenvironment a bi-specific molecule that comprises a cell-targeting domain that specifically binds to a cell-specific antigen expressed by the tumor cell and a Notch-binding domain that trans-binds to Notch expressed by a non-tumor cell in the tumor micro-environment, thereby inducing Notch signaling in the non-tumor cell.

As described above, the second cell that is targeted for trans Notch activation can be a stromal cell, an endothelial cell, or an immune cell. In some embodiments, the immune cell can be a monocyte and trans-binding of the bi-specific molecule to Notch on the monocyte promotes differentiation of the monocyte into a dendritic cell. In other embodiments, the trans-binding of the bi-specific molecule to Notch on the immune cell promotes differentiation to an M1 macrophage. In yet other embodiments, the trans-binding of the bi-specific molecule to Notch on the immune cell promotes conversion of myeloid derived suppressor cells from an anti-inflammatory state to a pro-inflammatory state.

The methods are applicable to therapeutic interventions for cancers characterized by aggregations of transformed cells, e.g., solid tumors. Induction of Notch signaling in cells within the tumor microenvironment promotes an immune-responsive, anti-tumor state (e.g., pro-inflammatory state). Such treatment can reduce the health of the tumor cells by overcoming the immunosuppression typical of tumor microenvironments and, thus, facilitating the body's own immune response against the transformed tumor cells. Additionally, because an immune-responsive state (e.g., pro-inflammatory) can weaken the tumor cells, the tumor cells can become more susceptible to other interventions. Thus, the disclosure encompasses the combination of the disclosed methods with additional therapeutic interventions, including the use of additional therapeutic against cancers.

As used herein, the terms “treatment,” “treating,” “therapeutic intervention,” and the like, refer to administering the bi-specific molecule for the purpose of obtaining an effect. The effect can be prophylactic in terms of completely or partially preventing a disease or symptom thereof and/or can be therapeutic in terms of achieving a partial or complete cure for a disease and/or symptoms of the disease. “Treatment,” as used herein, can include treatment of a tumor in a mammal, particularly in a human, and includes: inhibiting the disease, i.e., arresting or slowing its development; preventing recurrence of the disease; and/or relieving the disease, i.e., causing regression of the disease.

The term “subject” as used above in reference to the methods can refer to any animal with the target cell-type of interest. Subjects are typically mammals, and can include the non-limiting examples of primates (including, e.g., human, monkey, and the like), rodent (including, e.g., rat, mouse, guinea pig, and the like), dog, cat, horse, cow, pig, sheep, and the like.

The bi-specific molecule can be formulated and dosed for any appropriate route of administration. Furthermore, the administration of the bi-specific molecule, or a pharmaceutical composition containing the same, can also be administered in combination with other therapeutic interventions, including other anti-cancer therapeutics. In certain embodiments, at least one additional therapeutic and the disclosed bi-specific molecule as disclosed herein are administered concurrently to a subject. When administered in combination, each component can be administered at the same time or sequentially in any order at different points in time. Thus, each component can be administered separately but sufficiently closely in time so as to provide the desired therapeutic effect. Such additional therapeutic agents can be cytotoxic agents that are known to further inhibit or treat the cancer. Nonlimiting examples include aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, duocarmycin, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, saproin, tamoxifen, taxol, topotecan hydrochloride, trastuzumab, vinblastine, vincristine, vinorelbine tartrate, and the like.

In some embodiments, the additional therapeutic is an immune checkpoint inhibitor. For example, current checkpoint inhibitors are known that inhibit PD-1, PD-L1, or CTLA-4. In some embodiments, the immune checkpoint inhibits PD-1, such as a checkpoint inhibitor selected from Pembrolizumab (Keytruda), Nivolumab (Opdivo), and Cemiplimab (Libtayo). In some embodiments, the immune checkpoint inhibits PD-L1, such as a checkpoint inhibitor selected from Atezolizumab (Tecentriq), Avelumab (Bavencio), and Durvalumab (Imfinzi). In some embodiments, the immune checkpoint inhibits CTLA-4, such as Ipilimumab (Yervoy).

In some embodiments, the additional therapeutic is a composition comprising immune cells for an adoptive cell therapy. Adoptive cell therapy is a technique by which cells, typically immune cells, are cultivated in vitro and administered to a subject to improve the immune functionality of the subject against a particular target. The immune cells can be autologous or allogenic. Exemplary immune cells include T cells and NK cells. In some embodiments, the immune cells are modified or enhanced by culture environments applied in vitro. In some embodiments, the immune cells are genetically modified to enhance or confer a new functionality. For example, the cells (e.g., T cells or NK cells) can be genetically modified to express a chimeric antigen receptor (CAR) on the surface. The CAR typically contains an extracellular domain with enhanced affinity for an antigen of interest. The extracellular domain is linked to an intracellular signaling domain that activates the cell upon antigen binding. Such CAR-expressing cells can provide a powerful tool to combat pathogens and cancer cells because upon binding to the target antigen in vivo, the CAR-expressing cells undergo further expansion and activation to provide a type of “living drug” that can have a direct cytotoxic action against the target as well as influence the endogenous immune functionality through production of cytokines.

The bi-specific molecules encompassed by the present disclosure include the bi-specific reagents described in WO 2018/017827, incorporated herein by reference in its entirety. Elements of the bi-specific molecule are described below.

Notch-Binding Domain

As used herein, the term “Notch signaling” or other references to the function of Notch receptor refer to the cell-signaling cascade that occurs from the proteolytic cleavage of the expressed mature Notch receptors in a cell membrane. Notch receptors in mammals include Notch1, Notch2, Notch3, and Notch4, and homologs of which are known and readily ascertainable by persons of ordinary skill in the art for humans, rodents, and other species. For example, representative amino acid sequence for human Notch1 is provided in Genbank Accession No. P46531, which is incorporated herein by reference in its entirety. This is also set forth herein as SEQ ID NO:8. Other Notch receptors are well-known and readily identifiable. Illustrative, non-limiting examples of other Notch receptors include the following sequences: GenBank Accession No. AAH71562.2 (representative human Notch2), GenBank Accession No. AAB91371.1 (representative human Notch3), and GenBank Accession No. AAC63097.1 (representative human Notch4) (the sequence of each accession number is incorporated herein by reference). Similarly, Notch is also known and readily ascertainable in Drosophila, C. elegans, and other invertebrate species. Signaling of Notch receptor can be ascertained and monitored with any appropriate technique familiar in the art. For example, as described in more detail below, Notch signaling can be monitored by measuring downstream gene products resulting from Notch activation, such as Hest expression. Alternatively, reporter systems are available to indicate Notch signaling, such as the CHO-K1 Notch reporter system. See, e.g., Sprinzak, D., et al. “Cis-interactions between Notch and Delta generate mutually exclusive signalling states,” Nature 465(7294):86-90 (2010), incorporated herein by reference in its entirety.

As described herein, the bi-specific molecule can induce Notch signaling in an aggregation of cells that comprise at least a first cell-type that exhibits a substantially unique cell marker. The induction of Notch signaling refers to the relative increase of Notch signaling in a cell within the targeted aggregation, regardless of whether the signaling occurs in the cell with the substantially unique marker or not. The increase in Notch signaling is in a comparative scenario without application of the disclosed bi-specific molecule. This induction can be targeted, indicating that this induction of Notch signaling is realized primarily within the aggregation of cells (e.g., a tumor microenvironment) and occurs by virtue of the cell with induced Notch signaling being in close proximity with a cell expressing the marker such that the bi-specific molecule can trans-bind to the Notch receptor on the cell while simultaneously binding to the marker of interest on the neighboring cell. While the effect is ideally realized exclusively in aggregation of cells (e.g., tumor microenvironment), it will be understood that some effect can still occur in off-target cells or cell-types while remaining within the scope of the disclosure. Any induction in off-target effects compared to non-targeted therapies still confers a utility of reducing toxicity and side-effects and, thus, is a desired result achieved by the present disclosure. In some embodiments, the disclosed bi-specific molecule does not substantially induce Notch signaling in off-target cells or cell-types, e.g., cells that do not reside in the target aggregation of cells (e.g., tumor microenvironment).

The Notch binding domain of the bi-specific molecule can comprise a Notch binding domain of any Notch receptor ligand. Similarly, the Notch binding domain of the bi-specific molecule can be derived from a Notch binding domain of any Notch receptor ligand as long as the derivative retains Notch binding affinity sufficient to measurably inhibit Notch proteolysis and subsequent signaling. As used herein, the term “derived” indicates that the derivative is obtained from the source molecule or sequence, but can contain changes (e.g., substitution, deletions, additions) from the source molecule or sequence. Typically, the derivative includes substantially the same amino acid sequence as the source molecule. “Substantially the same” in certain contexts is described in terms of % sequence identity, e.g., a variant that is at least 80% identical to a parental sequence and having one or more substitutions, as determined using standard and accepted methodologies in the art. In some embodiments, the derivative can have an amino acid sequence that is at least 80%, 81%, 82%, 83%, 84%, 85%, 86%, 87%, 88%, 89%, 90%, 91%, 92%, 93%, 94%, 95%, 96%, 97%, 98%, and 99% identical to a parental sequence. The derivative can also contain chemical modifications, such as to one or more amino acid residues, within the original source sequence.

The indicated Notch receptor ligand includes any canonical or noncanonical ligand to mammalian Notch receptor (e.g., a ligand to Notch1, Notch2, Notch3, or Notch4 receptor). Such ligands can be, or can be derived from, mammalian Notch receptor ligands. As indicated above, the canonical Notch ligands in mammals include Jagged proteins (e.g., Jagged1and Jagged2) and Delta proteins (e.g., DLL1, DLL3, DLL4; where DLL is an acronym for Delta Like Ligand), each of which are well-known and are contemplated and encompassed by this disclosure. As non-limiting examples, representative canonical Notch ligand sequences comprise sequences set forth in GenBank Accession No. AAC51731 (Jagged1), GenBank Accession No. AAD15562 (Jagged2), GenBank Accession Nos. ABC26875 or NP005609 (DLL1), GenBank Accession Nos. NP_982353.1 or NP_058637.1 (DLL3), and NP_061947.1 (DLL4) (the sequence of each accession number incorporated herein by reference), homologs, or functional (Notch binding) variants, fragments, or derivatives thereof. These canonical ligands, collectively referred to as DSL ligands, typically contain an N-terminal region, a DSL domain, and at least the first two EGF-like repeats, which are necessary for interaction with EGF repeats 11 and 12 of Notch receptors. Accordingly, in some embodiments, the Notch binding domain comprises an extracellular domain of a Delta protein or a Jagged protein, such as vertebrate (e.g., mammalian) or invertebrate Delta or Jagged proteins, as described herein. A 2.3 angstrom resolution crystal structure of interacting regions of Notch1-DLL4 indicates the structural components of the ligand-receptor complex important for binding. See Luca, V. C., et al., “Structural Basis for Notch1 Engagement of Delta-Like 4,” Science 347(6224):847-853 (2015).

In some embodiments, the Notch binding domain can include polypeptide sequences with one or more mutations in a wild-type sequence resulting in modified affinity for the Notch receptor. Accordingly, a person of ordinary skill in the art can readily identify minimal Notch binding domains from known or putative Notch ligands. Luca, et al., (2015), supra, which is incorporated herein in its entirety, further discloses modifications in the wild-type DLL4 that enhance binding affinity to the receptor, thus further illuminating required and critical domains in a canonical Notch ligand required for binding to the Notch receptor. For example, as demonstrated in the E12 variant of rat DLL4 disclosed in Luca, et al. (2015), mutations of G28S, F107L, L206P, N118I, I143F, H194Y, K215E, individually or in any combination, can enhance affinity of binding. Accordingly, in an illustrative, non-limiting embodiment, the Notch binding domain can comprise an amino acid sequence with at least 80% (such as about 80%, 81%, 82%, 83%, 84%, 85%, 86%, 87%, 88%, 89%, 90%, 91%, 92%, 93%, 94%, 95%, 96%, 97%, 98%, and 99%) sequence identity to the sequence set forth in SEQ ID NO:2. SEQ ID NO:2 is a wild-type polypeptide sequence of a rat DLL4 fragment corresponding to the MNNL to EGF2 domains (i.e., amino acid positions 27 to 283) of the full-length precursor. The full length rat DLL4 precursor is set forth herein as SEQ ID NO:1. In some embodiments, the Notch binding domain comprises a polypeptide with a sequence that includes at least one substitution at an amino acid position selected from: 28, 43, 52, 96, 107, 118, 143, 146, 183, 194, 206, 215, 223, and 257 (the positions are numbered with respect to positions within the reference sequence set forth in SEQ ID NO:1 and corresponding homologous positions in other DLL proteins can be readily ascertained by alignment). In certain embodiments, the at least one substitution enhances affinity. In some embodiments, the at least one substitution is selected from: G28S, MN43I, P52S, S96I, F107L, N1181, I143F/T, Q146K, S183N, H194Y, L206P, K215E, L223R, and N257K, or a similar substitution at a corresponding amino acid residue in a homologous sequence. In some instances, the high affinity Notch receptor ligand comprises at least 2, 3, 4, 5, 6, 7, 8, 9, 10 or more of the substitutions set forth above. Any combination of substitutions as set forth above is contemplated. Examples of specific combinations of substitutions include, but are not limited to: (i) P52S, F107L, L206P; (ii) F107L, L206P, N257K; (iii) F107L, L223R, N257K; (iv) G28S, M43I, F107L, N118I; (v) G28S, F107L, N118I, Q146K, H194Y, L206P, K215E; (vi) G28S, F107L, N118I, I143F, H194Y, L206P, K215E; (vii) G28S, M43I, S96I, N118I, I143T, S183N, H194Y, L206P, K215E; (viii) G28S, F107L, L206P; and (ix) G28S, F107L, L206P, N257K (or a similar substitution at a corresponding amino acid residue in a homologous sequence). Also disclosed in Luca, et al. (2015), mutations to Jagged proteins could be mapped to the sequence of DLL4 indicating important residues on this ligand for contact and binding on the Notch receptor. Thus, the Notch binding domain can comprise an amino acid sequence with at least 80% (such as about 80%, 81%, 82%, 83%, 84%, 85%, 86%, 87%, 88%, 89%, 90%, 91%, 92%, 93%, 94%, 95%, 96%, 97%, 98%, and 99%) sequence identity to the sequence set forth in SEQ ID NO:5, which sets forth the amino acid sequence corresponding to the amino acids 32 to 295 of the full wild-type rat Jaggedl polypeptide. The full wild-type rat Jagged1 polypeptide sequence is set forth in SEQ ID NO:4. In additional embodiments, the Notch binding domain can comprise at least one substitution at an amino acid position selected from 100 and 182, with reference to positions in SEQ ID NO:4 (although not requiring the entire sequence; homologous positions in other DLL proteins can be readily ascertained by alignment). In certain embodiments, the at least one substitution is selected from: P100H, Q183P, and a combination thereof. Alternatively, in homologous sequences, the at least on substitution can be at the corresponding amino acid residue position(s) in the homologous sequence.

In other embodiments, the Notch binding domain can comprise an amino acid sequence with at least 80% (such as about 80%, 85%, 90%, 91%, 92%, 93%, 94%, 95%, 96%, 97%, 98%, 99%, or 100%) sequence identity to the sequence set forth in SEQ ID NO:6 or 7, which set forth the amino acid sequence of the extracellular Notch-binding regions of representative human Jagged2 (Genbank Accession No. AAD15562.1) and human Delta like 1 (DLL1; Genbank Accession No. NP005609.3), respectively. In view of the above structural studies and other available data, persons of ordinary skill in the art can readily ascertain permissible variations in the reference sequences that still result in functional binding to the Notch receptors.

In addition to the Notch binding domains of canonical Notch ligands, the Notch binding domain of the bi-specific molecule can comprise a Notch binding domain (or a Notch-binding derivative or fragment thereof) of any non-canonical Notch receptor ligand, such as the binding domain of Dlk1, Dlk2, DNER, EGFL 7, and F3/contactin, which are more typically involved in cis-inhibition. See, e.g., Hu, Q., et al., “F3/contactin acts as a functional ligand for Notch during oligodendrocyte maturation,” Cell 115(2):163-175 (2003); Schmidt, M. H., et al., “Epidermal growth factor-like domain 7 (EGFL7) modulates Notch signalling and affects neural stem cell renewal,” Nat Cell Biol 11(7):873-880 (2009); and D'Souza, B., et al., “Canonical and non-canonical Notch ligands,” Curr Top Dev Biol 92:73-129 (2010), each of which is incorporated herein by reference in its entirety. The fragments or derivatives retain the ability to bind the target Notch receptor. In some embodiments, the derivative can comprise an amino acid sequence with at least 80% (such as about 80%, 81%, 82%, 83%, 84%, 85%, 86%, 87%, 88%, 89%, 90%, 91%, 92%, 93%, 94%, 95%, 96%, 97%, 98%, and 99%) of the sequence of the source Notch binding domain of the non-canonical Notch receptor ligand.

While the above description included examples of rat or human Notch ligands, it will be appreciated that the indicated mammalian sources for Notch ligands can include the non-limiting examples of primates (including, e.g., human, monkey, and the like), rodent (including, e.g., rat, mouse, guinea pig, and the like), dog, cat, horse, cow, pig, sheep, and the like. Non-mammalian Notch ligands, such as Drosophila Serrate and Delta, are also well-known and are encompassed by the present disclosure. As indicated, the Notch signaling system is highly conserved and, thus, homologous sequence positions among the Notch receptors and respective Notch ligands are readily ascertainable by persons of ordinary skill in the art.

In addition to Notch binding domain comprising or being derived from a known Notch receptor ligand, as described above, the Notch binding domain of the disclosed bi-specific molecule can also be or comprise an affinity reagent designed to specifically bind a Notch receptor. As used herein, “affinity reagent” refers to any molecule that can bind a target antigen, in this case a Notch receptor, with a specific affinity (i.e., detectable over background). Exemplary, non-limiting categories of affinity reagent include antibodies, an antibody-like molecule (including antibody derivatives and antigen (i.e., Notch)-binding fragments thereof), peptides that specifically interact with a particular antigen (e.g., peptibodies), antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, Curr. Opin. Biotechnol. 22:849-857, 2011, and references cited therein, each incorporated herein by reference in its entirety]), aptamers, or a functional Notch-binding domain or fragment thereof. These affinity reagents are described in more detail below in the “Additional definitions” section. Such affinity reagents can be generated through application of routine techniques based on the known Notch targets described above.

As used herein, the term “specifically bind” or variations thereof refer to the ability of the affinity reagent component to bind to the antigen of interest (e.g., Notch receptor or, as described below, the antigen characteristic of the cell-type of interest), without significant binding to other molecules, under standard conditions known in the art. The antigen-binding molecule can bind to other peptides, polypeptides, or proteins, but with lower affinity as determined by, e.g., immunoassays, BlAcore, or other assays known in the art. However, affinity reagent preferably does not substantially cross-react with other antigens.

In some embodiments, the Notch-binding domain of the bi-specific molecule, whether derived from a Notch-binding domain of a Notch receptor ligand (e.g., DLL4) or from an affinity reagent described above (e.g., an antibody or antibody-like molecule), has a binding affinity sufficient for binding the Notch receptor on a cell (e.g., of the second cell described herein) when sufficiently targeted by a high affinity cell-targeting domain. Thus, in some embodiments, the Notch-binding domain of the bi-specific molecule has a binding affinity within a range characterized by a dissociation constant (K_{d}) from about 100 nM (lower binding affinity) to about 0.1 nM (higher binding affinity). For example, the Notch-binding domain has a binding affinity for the Notch receptor characterized by (K_{d}) of about 100 nM 90 nM, 80 nM, 70 nM, 60 nM, 50 nM, 40 nM, 30 nM, 20 nM, 10 nM, 5 nM, 1 nM, and 0.1 nM. Exemplary (K_{d}) ranges include from about 100 nM to about 40 nM, from about 80 nM to about 20 nM. Other exemplary (K_{d}) ranges include from about 60 nM to about 1 nM, from about 80 nM to about 60 nM, from about 70 nM to about 50 nM, from about 60 nM to about 40 nM, from about 50 nM to about 30 nM, from about 40 nM to about 20 nM, from about 30 nM to about 10 nM, from about 20 nM to about 1 nM, from about 10 nM to about 0.01 nM, and any subrange therein. While sufficient binding affinity between the Notch-binding domain of the bi-specific molecule and the Notch receptor is required to functionally induce trans-activation in the second cell, described herein, the affinity should not be so high as to induce indiscriminate binding of the bi-specific molecule throughout the body of a subject if given a systemic administration of the bi-specific molecule. Such systemic Notch binding would counteract the intended cell-specific functionality of the disclosed bi-specific molecule. Instead, cell-specificity is conferred by the cell-targeting domain, which can have a similar affinity, higher affinity, or lower affinity for an antigen characteristic of the first cell-type of interest, which is described below, to provide optimal targeting.

Cell Targeting Domain

As indicated above, the cell-targeting domain specifically binds to an antigen characteristic of the first cell-type in the aggregation of cells (e.g., tumor cells or non-tumor cells in a tumor microenvironment). In other embodiments, the cell-targeting domain specifically binds to an extracellular antigen or substrate present in the tumor microenvironment, such as collagen. The cell-targeting domain can bind to the antigen with an affinity that similar to, greater than, or less than the binding affinity of the Notch-binding domain for the Notch receptor, as described above. The particular affinity of the cell-targeting domain can be adjusted to optimize targeting capability and reduce off-target binding and Notch activation. In some cases, the cell-targeting domain typically binds to the antigen characteristic of the cell-type of interest with an affinity that is at least about 2 times, 3 times, 4 times, 5 times, 6 times, or 7 times greater than the binding affinity of the Notch-binding domain for the Notch receptor. In some instances, the binding affinity of the cell-targeting domain for the antigen characteristic of the cell-type of interest is at least an order of magnitude greater than the binding affinity of the Notch binding domain for a Notch receptor. For example, the dissociation constant (K_{d}) characterizing the affinity of the cell-targeting domain for the antigen characteristic of the cell-type of interest can be about 50 nM, 40 nM, 30 nM, 20 nM, 10 nM, 5 nM, 1 nM, 0.75 nM, 0.5 nM, 0.1 nM, 0.05 nM, 0.01 nM, 0.005 nM, and 0.001 nM, or even smaller. Typical (K_{d}) ranges characterizing the binding affinity of the cell-targeting domain for the antigen characteristic of the cell-type of interest include from about 30 nM to about 10 nM, from about 20 nM to about 1 nM, from about 10 nM to about 0.1 nM, from about 0.5 nM to about 0.05 nM, and from about 0.1 nM to about 0.001 nM, or even lower, or any subrange therein.

The cell-targeting domain comprises an affinity reagent designed to specifically bind to an antigen characteristic of the first cell-type in the aggregation of cells (e.g., tumor cells, or non-tumor cells, or an extracellular substrate in a tumor microenvironment). In this context, the term “affinity reagent” refers to any molecule that can bind the antigen characteristic of the cell-type of interest with a specific affinity (i.e., detectable over background). As with the above description with respect to the Notch-binding domain, exemplary, non-limiting categories of affinity reagent include antibodies, an antibody-like molecule (including antibody derivatives and antigen (i.e., cell-specific antigen)-binding fragments thereof), peptides that specifically interact with a particular antigen (e.g., peptibodies), antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, Curr. Opin. Biotechnol. 22:849-857, 2011, and references cited therein, each incorporated herein by reference in its entirety]), aptamers, or a functional Notch-binding domain or fragment thereof. Again, these affinity reagents are described in more detail below in the “Additional definitions” section.

The antigen characteristic of a cell-type of interest can be any relevant antigen known to be predominantly present and accessible on a target cell, i.e., the first cell-type in the aggregation of cells (e.g., tumor cells or non-tumor cells, or an extracellular substrate in a tumor microenvironment) or that is otherwise present within the tumor microenvironment. The chosen antigen is preferably substantially absent or reduced (e.g., expressed at lower levels) in non-target cells or outside of the tumor microenvironment so as to confer specific and preferential binding by the bi-specific molecule for the first cell-type (or product thereof) in the aggregation of cells (e.g., tumor cells or non-tumor cells, or an extracellular substrate in a tumor microenvironment), and thus does not substantially bind to cells not in the aggregation of cells. Thus, the term antigen “characteristic” of a cell-type of interest is not intended to indicate that the antigen is completely exclusive to the first cell-type in the aggregation of cells, but rather the expression or elevated level of expression is at least typical of the target cell-type and distinguishes that cell-type from the majority of other cells. As indicated above, any targeting that reduces indiscriminate binding of the molecule to Notch receptors systemically throughout the body is advantageous for therapeutic interventions. In some cases, the binding affinity of the Notch binding domain is such that binding to a Notch receptor will first require the cell-targeting domain to bind to its cognate antigen.

Persons of ordinary skill in the art can readily select any appropriate antigen for the design and implementation of the cell-targeting domain according to the vast cataloguing of characteristic target cell biomarkers known in the art.

In some embodiments, the antigen is a cell surface biomarker for a cancer or tumor cell. As used herein, the term “cancer” refers to cells which exhibit autonomous, unregulated growth, such that they exhibit an aberrant growth phenotype characterized by a significant loss of control over cell proliferation and tend to form aggregations or tumors with unique microenvironments compared to healthy tissues. Cells of interest for detection, analysis, or treatment in the present application include precancerous (e.g., benign), malignant, pre-metastatic, metastatic, and non-metastatic cells. Many types of cancers and substantially unique markers thereof are known to those of skill in the art, including solid tumors such as carcinomas, sarcomas, glioblastomas, melanomas, lymphomas, myelomas, and the like. Illustrative cancers or cancer cell types encompassed by the present disclosure include but are not limited to ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer. In some embodiments, the cancer cell is selected from breast cancer cell, prostate cell, lung cancer cell, glioblastoma, colorectal cancer cell, cervical cancer cell, melanoma cancer cell, pancreatic cancer cell, esophageal cancer cell, and the like.

Cancer antigens can be, for example, tumor specific or tumor associated antigens that are known in the art. Exemplary antigens that are characteristic of various cancers and their qualifications as determinants of cancer cells are discussed widely in the literature. For example, see Cheever, Martin A., et al., “The prioritization of cancer antigens: a national cancer institute pilot project for the acceleration of translational research,” Clinical Cancer Research 15(17):5323-5337 (2009), incorporated herein by reference in its entirety. In some embodiments, the antigen characteristic of a cell-type of interest can be a cell surface marker of any cancer or tumor type of interest. In a few illustrative, non-limiting embodiments the antigen characteristic of a cell-type of interest (i.e., the first cell-type) is CD33, CD326, CD133, or mesothelin.

Relevant antigens that are characteristic of the cancer cells of interest (i.e., the first cell-type in the methods described herein) are known and domains that specifically bind to such antigens are available or can be readily produced for incorporation into the disclosed bi-specific molecule. An illustrative, non-limiting example of an antigen characteristic of a target cell-type is the cell-surface marker CD33. Accordingly, as described in more detail below and in WO 2018/017827, incorporated herein by reference in its entirety, this antigen was targeted using a bi-specific molecule referred to as DLL4_{E12}-αCD33 scFv fusion molecule, where the aCD33 scFv served as the cell-targeting domain to specifically target tumor cells known to express CD33. Thus, one illustrative cell-targeting domain can have the amino acid sequence set forth in SEQ ID NO:9, or a functional variant thereof that binds to CD33. Such a functional variant of the CD33 binding domain can comprise a sequence with at least 80% (such as about 80%, 85%, 90%, 91%, 92%, 93%, 94%, 95%, 96%, 97%, 98%, 99%, or 100%) sequence identity to the sequence set forth in SEQ ID NO:9.

In another illustrative, non-limiting example, the antigen characteristic of a target cell-type (i.e., the first cell-type) is mesothelin. Mesothelin (MSLN) is a 40 kDA protein that first gained attention as a tumor marker and potential therapeutic target for its overexpression in many solid tumors, including mesothelioma, ovarian carcinoma, and pancreatic adenocarcinoma. It is normally expressed by mesothelial cells lining the pleura, pericardium, and peritoneum, as well as in reproductive organs, and is anchored to the external cell surface by glycophosphatidylinositol (GPI). The biological function of cell surface MSLN is unknown, although hypothesized functionality includes a role in cell adhesion. See, e.g., WO 2020/092631, incorporated herein by reference in its entirety, provides additional description to the mesothelin antigen and its use as an antigen to target therapeutic payloads.

As indicated above, the antigen targeted by the cell-targeting domain can target antigens expressed on non-tumor cells within the tumor microenvironment. Additionally, the antigens do not necessarily have to be expressed on the surface of the first cell, but rather can be a product thereof and have sufficiently high presence in the tumor microenvironment to facilitate trans-binding of Notch on the second cell-type within the tumor microenvironment. Such antigens include extracellular substrates, such as collagen.

Fusion Constructs, Linkers

In one embodiment, the cell-targeting domain and the Notch-binding domain are disposed consecutively, in any order or orientation, within the bi-specific molecule. In an alternative embodiment, the cell-targeting domain and the Notch-binding domain, in any order or orientation, are joined by at least an intervening flexible linker domain. The linker domain functions as a spacer to allow each domain sufficient space to assume its natural three-dimensional shape without requiring significant adjustment, thus allowing freedom to contact and bind their corresponding targets without mutual interference. The linker can be of sufficient length and flexibility to allow independent movement of each domain, thus maximizing their potential to locate and bind their respective targets. The linker can be a synthetic polypeptide sequence, which is typically between about four and about 40 amino acids in length (e.g., about 5, 10, 15, 20, 25, 30, 35, 40 amino acids), although it can be longer, and can be part of an expressed fusion construct. The linker is typically designed to avoid significant formation of rigid secondary structures that could reduce the flexibility or distance provided between the proximate components. Thus, the linker is designed to provide a linear or alpha-helical structure. Such linkers are commonly used and are well-understood in the art. An illustrative example of a linker is a 15 amino acid residue linker with 3× repeats of the sequence GSGSGSGSGS, which was utilized in a specific embodiment described in more detail below.

In some embodiments, the bi-specific molecule is a fusion polypeptide and the cell-targeting domain and Notch binding domain are polypeptides that do not naturally occur together. The term “fusion” in the context of a fusion protein indicates that the overall protein or polypeptide contains a non-naturally occurring polypeptide sequence. The fusion protein combines to two or more existing polypeptides or polypeptide fragments (i.e., the distinct cell-targeting and Notch-binding domains, and optionally an intervening linker), from the same or different source proteins, in a chimeric polymer where the polypeptides (or fragments) do not naturally occur together in that manner. Methods of producing fusion proteins are well known. For example, nucleic acids encoding the different polypeptide components of the fusion protein can be generated and amplified using PCR and assembled into an expression vector in the same reading frame (with or without intervening sequence encoding a linker) to produce a fusion gene. The expression vector can be transformed into any appropriate expression system, such as prokaryotic or eukaryotic cells, which can then express the protein. See, e.g., such standard references as Coligan, Dunn, Ploegh, Speicher and Wingfield, “Current Protocols in Protein Science” (1999), Volume I and II (John Wiley & Sons Inc.); Sambrook et al., “Molecular Cloning: A Laboratory Manual” (1989), 2nd Edition (Cold Spring Harbor Laboratory Press); and Prescott, Harley and Klein. “Microbiology” (1999), 4th Edition (WBC McGraw Hill), each incorporated herein by reference in its entirety. One exemplary approach for creating fusion proteins is described in more detail in the below examples. In another embodiment, the fusion protein can be created by linking the two polypeptide fragments corresponding to the separate cell-targeting and Notch-binding domains. Each of these components can be separately generated or obtained independently from one another by any known and conventional technique. The components can subsequently be fused or linked to one another by chemical means. For example, each component can have complementary binding partner moieties such that they will form strong mutual bonds, thereby linking their respective components to produce the fusion protein. The linker moieties can be homobifunctional or heterobifunctional. An illustrative, nonlimiting example of such chemical binding partner components include having one component (e.g., the cell-targeting domain) include biotin and the other component (e.g., Notch binding domain) include (strept)avidin, or vice versa. The biotin and (strept)avidin moieties will form high-affinity bonds, thereby linking, or “fusing,” the components to result in the fusion protein. Other common linking chemistries can also be used, such as, for example, gluteraldehyde, and the like.

In some embodiments, the bi-specific molecule is isolated. In this context, the term “isolated” indicates that the bi-specific molecule, e.g., in the form of a fusion protein, has been produced through human intervention and has been substantially separated from the materials co-existing in the production environment, such as the intra-cellular organelles and proteins in a cell expression system. In contrast, a naturally expressed protein in cell is not “isolated.”

As described in more detail in WO 2018/017827, incorporated herein by reference in its entirety, a bi-specific molecule, referred to as DLL4_{E12}-αCD33 scFv fusion molecule, with a sequence set forth in SEQ ID NO:10, was generated and successfully applied to specifically inhibit Notch signaling on CD33⁺ cells. Accordingly, in some embodiments, the bi-specific molecule comprises a sequence with at least 80% (such as about 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the sequence set forth in SEQ ID NO:10.

In one embodiment, the bi-specific molecule is or comprises SEQ ID NO:10.

Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); Ausubel, F.M., et al. (eds.), Current Protocols in Molecular Biology, John Wiley & Sons, New York (2010); and Coligan, J.E., et al. (eds.), Current Protocols in Immunology, John Wiley & Sons, New York (2010) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, in some embodiments “about” can refer to a number within a range of 10%, 9%, 8%, 7%, 6%, 5%, 4%, 3%, 2%, or 1% above or below the indicated reference number.

“Percent sequence identity” or grammatical equivalents means that a particular sequence has at least a certain percentage of amino acid residues identical to those in a specified reference sequence using an alignment algorithm. An example of an algorithm that is suitable for determining sequence similarity is the BLAST algorithm, which is described in Altschul, et al., J. Mol. Biol. 215:403-410 (1990). Software for performing BLAST analyses is publicly available through the National Center for Biotechnology Information (NCBI) website.

The term “wild-type,” “wild-type,” “WT” and the like refers to a naturally-occurring polypeptide or nucleic acid sequence, i.e., one that does not include a man-made variation.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having cancer. Subjects may be human, but also include other mammals, particularly those mammals useful as laboratory models for human disease, e.g., mouse, rat, dog, non-human primate, etc.

“Treating” can refer to any indicia of success in the treatment or amelioration or prevention of a cancer, including any objective or subjective parameter such as abatement;

remission; diminishing of symptoms or making the disease condition more tolerable to the patient; slowing in the rate of degeneration or decline; or making the final point of degeneration less debilitating.

The treatment or amelioration of symptoms can be based on objective or subjective parameters; including the results of an examination by a physician. Accordingly, the term “treating” includes the administration of the compounds or agents of the present disclosure to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

As indicated above, certain embodiments of the bi-specific molecule comprise an affinity reagent that serves as the cell-targeting domain and/or the Notch binding domain. In some embodiments, the indicated affinity reagent is an antibody. As used herein, the term “antibody” encompasses antibodies and antibody fragments thereof, derived from any antibody-producing mammal (e.g., mouse, rat, rabbit, and primate including human), that specifically bind to an antigen of interest (e.g., Notch or a cell-type specific antigen). Exemplary antibodies multi-specific antibodies (e.g., bispecific antibodies); humanized antibodies; murine antibodies; chimeric, mouse-human, mouse-primate, primate-human monoclonal antibodies; and anti-idiotype antibodies. The antigen-binding molecule can be any intact antibody molecule or fragment thereof (e.g., with a functional antigen-binding domain).

An antibody fragment is a portion derived from or related to a full-length antibody, preferably including the complementarity-determining regions (CDRs), antigen binding regions, or variable regions thereof. Illustrative examples of antibody fragments and derivatives useful in the present disclosure include Fab, Fab′, F(ab)₂, F(ab′)₂ and Fv fragments, nanobodies (e.g., V_{H}H fragments and V_{NAR} fragments), linear antibodies, single-chain antibody molecules, multi-specific antibodies formed from antibody fragments, and the like. Single—chain antibodies include single-chain variable fragments (scFv) and single-chain Fab fragments (scFab). A “single-chain Fv” or “scFv” antibody fragment, for example, comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Single-chain antibodies can also include diabodies, triabodies, and the like. Antibody fragments can be produced recombinantly, or through enzymatic digestion.

The above affinity reagent does not have to be naturally occurring or naturally derived, but can be further modified to, e.g., reduce the size of the domain or modify affinity for the Notch (or cell-specific antigen) as necessary. For example, complementarity determining regions (CDRs) can be derived from one source organism and combined with other components of another, such as human, to produce a chimeric molecule that avoids stimulating immune responses in a subject.

Production of antibodies or antibody-like molecules can be accomplished using any technique commonly known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art. Once a monoclonal antibody is identified for inclusion within the bi-specific molecule, the encoding gene for the relevant binding domains can be cloned into an expression vector that also comprises nucleic acids encoding the remaining structure(s) of the bi-specific molecule.

Antibody fragments that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present invention can also be generated using various phage display methods known in the art.

As used herein, the term “aptamer” refers to oligonucleic or peptide molecules that can bind to specific antigens of interest. Nucleic acid aptamers usually are short strands of oligonucleotides that exhibit specific binding properties. They are typically produced through several rounds of in vitro selection or systematic evolution by exponential enrichment protocols to select for the best binding properties, including avidity and selectivity. One type of useful nucleic acid aptamers are thioaptamers, in which some or all of the non-bridging oxygen atoms of phophodiester bonds have been replaced with sulfur atoms, which increases binding energies with proteins and slows degradation caused by nuclease enzymes. In some embodiments, nucleic acid aptamers contain modified bases that possess altered side-chains that can facilitate the aptamer/target binding.

Peptide aptamers are protein molecules that often contain a peptide loop attached at both ends to a protamersein scaffold. The loop typically has between 10 and 20 amino acids long, and the scaffold is typically any protein that is soluble and compact. One example of the protein scaffold is Thioredoxin-A, wherein the loop structure can be inserted within the reducing active site. Peptide aptamers can be generated/selected from various types of libraries, such as phage display, mRNA display, ribosome display, bacterial display and yeast display libraries.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

### Example 1

The following is a description of a study characterizing the use of a bi-specific reagent that binds to CD33 and to Notch receptor in vivo. The bi-specific reagent was originally designed and used to inhibit Notch signaling in a target cell by promoting cis-binding. See, WO 2018/017827, incorporated herein by reference in its entirety. In the present study, the bi-specific reagent was assessed in vivo and was surprisingly found to increase Notch signaling in specific circumstances. This study illustrates an exemplary embodiment of the methods disclosed in the present disclosure.

Real-time tumor imaging shows that the bi-specific reagent activates Notch in vivo.

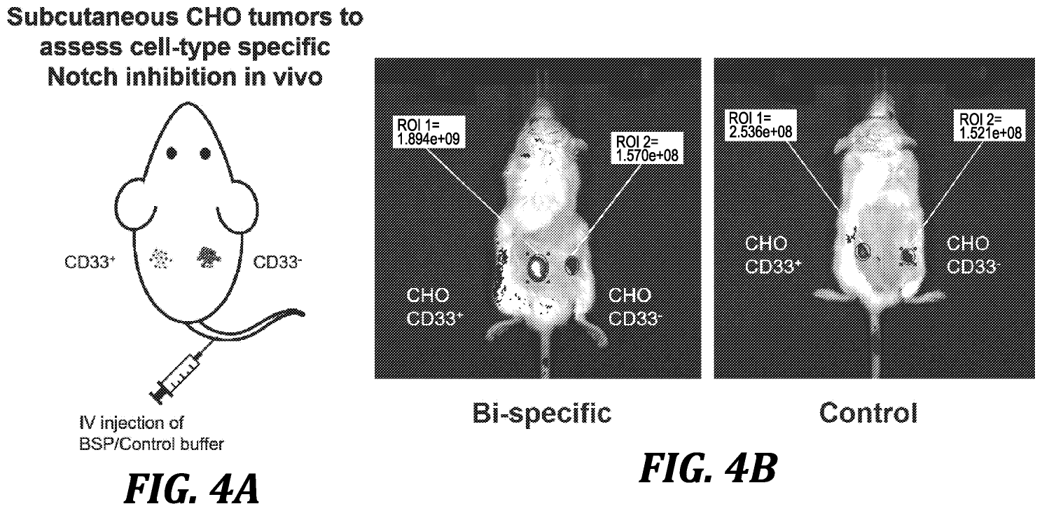

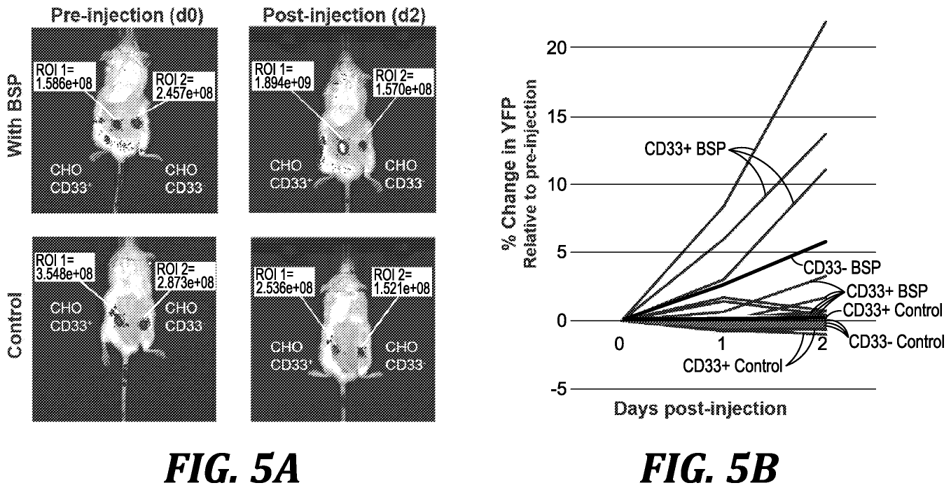

Chinese Hamster Ovary (CHO) cells were engineered to contain a Notch activation reporter (Sprinzak D, et al. Nature. 2010;465(7294):86-90). In this line, the Notch1 intracellular domain is replaced with yeast Ga14. Upon Notch receptor stimulation by trans-presented Notch ligand, Gal4 is released allowing for induction of a UAS-driven YFP reporter, a fluorophore easily distinguished. 1×10⁷ CHO cells that were further engineered to be CD33⁺ or CD33⁻ were separately subcutaneously injected into the flank of sub-lethally irradiated NOD/SCID gamma null (NSG) mice, producing a CHO-CD33⁺ tumor on the left and a CHO-CD33⁻ tumor on the right. Following the detection of solid tumors, Notch activation levels were assessed (by detecting YFP expression) prior to treatment with DLL4_{E12}-αCD33 scFv (the bi-specific protein (BSP) or “reagent”) using IVIS in vivo tumor imaging system (Perkin Elmer). 1.5 mg of DLL4_{E12}-αCD33 scFv or buffer control were then intravenously injected into each mouse. See generally WO 2018/017827 for the methodology, incorporated herein by reference in its entirety. At day 1 and day 2 post-injection, the bi-specific protein reagent (BSP) was assessed for whether it led to changes in Notch activation (as determined by YFP expression) in each tumor sub-type using IVIS. See FIGS. 4A-5B. As graphically illustrated in FIGS. 5A and 5B, administration of BSP resulted in significant increase of Notch expression in the tumors that had CD33⁺ cells.

In the context of a solid tumor, the prototypic bi-specific reagent activates Notch due to capture and trans-presentation within the tumor mass.

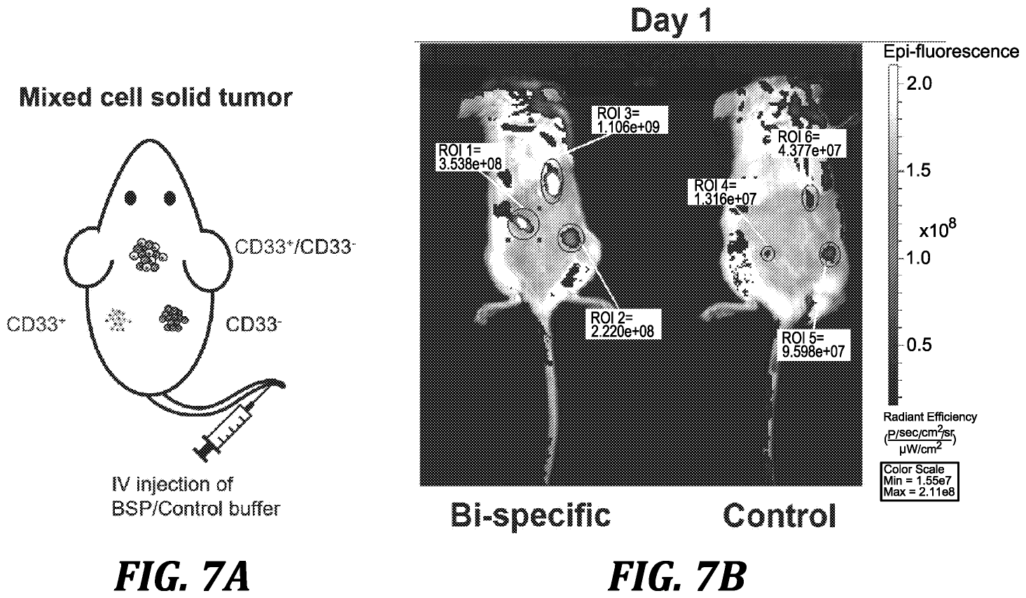

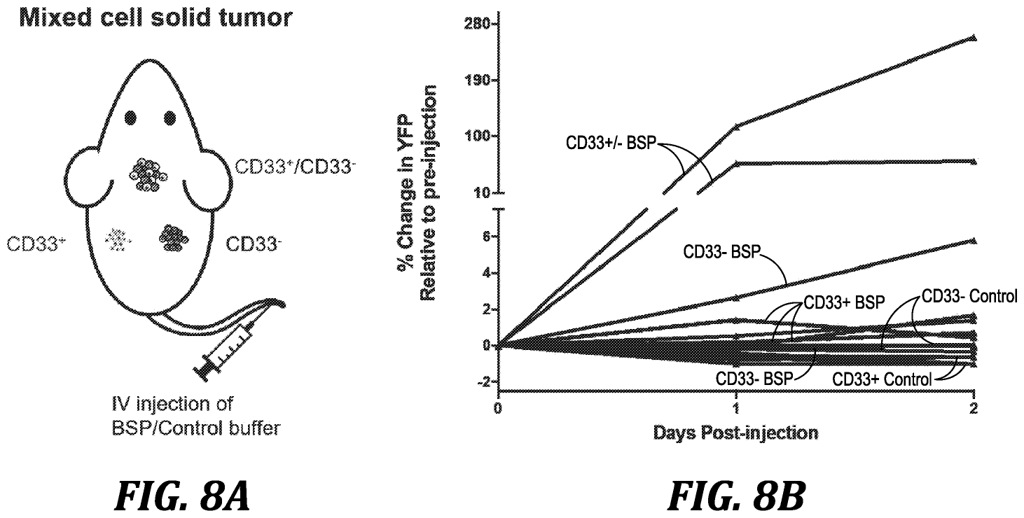

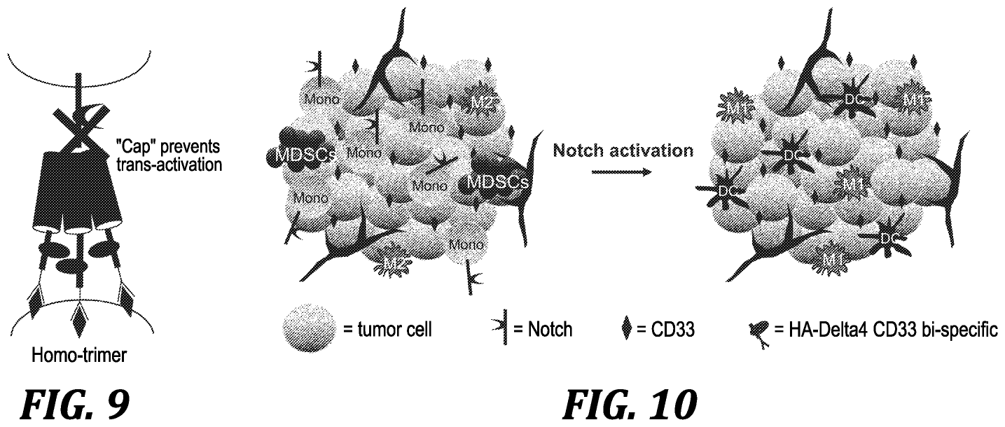

1×10⁷ Chinese Hamster Ovary (CHO)-CD33⁺ or CHO-CD33⁻ cells separately or in combination with CHO-CD33⁺/CD33⁻ (2×10⁷ total cells) were subcutaneously injected into the flank of sub-lethally irradiated NOD/SCID gamma null (NSG) mice, producing a CHO-CD33⁺ tumor on the left, a CHO-CD33⁻ tumor on the right, and a CHO-CD33⁺/CD33⁻ in the middle. Following the detection of solid tumors, Notch activation levels were assessed (by detecting YFP expression) prior to treatment with DLL4_{E12}-αCD33 scFv using IVIS in vivo tumor imaging system (Perkin Elmer). 1.5 mg of DLL4_{E12}-αCD33 scFv or buffer control were then intravenously injected into each mouse. At day 1 and day 2 post-injection, the bi-specific reagent was assessed for whether it led to changes in Notch activation (YFP) in each tumor sub-type using IVIS. See FIGS. 7A-8B. Treatment with bi-specific was observed to lead to a marginal increase (1.5×) in Notch activation in CD33+ expressing cells, likely due to the bi-specific functioning as an inhibitor when presented in cis on the CD33+ target cells. However, greater than a 10-fold increase in Notch activation was observed in the mixed cell setting. As graphically illustrated in FIGS. 8A and 8B, individuals with tumors that combined CD33⁺ and CD33⁻ cells had a significantly increased Notch signaling after administration of the BSP indicating maximized induction of Notch signaling. These data demonstrate that targeting the bi-specific protein reagent to CD33⁺ cells creates a Notch activating field capable of activating Notch on adjacent CD33⁻ cells.

Development of a murine CD33⁺ cell line capable of generating solid tumors in vivo.

4T1 Mammary Carcinoma cells (4T1s) expressing human ROR1 and Firefly luciferase-GFP were transduced with lentivirus encoding human CD33 and co-expressing mCherry. 4T1-CD33 cells were isolated by cell sorting and limit dilution cloning.

Bi-specific targeting 4T1 mammary carcinoma cells alters immuno-phenotype and gene expression of tumor-associated myeloid cells.

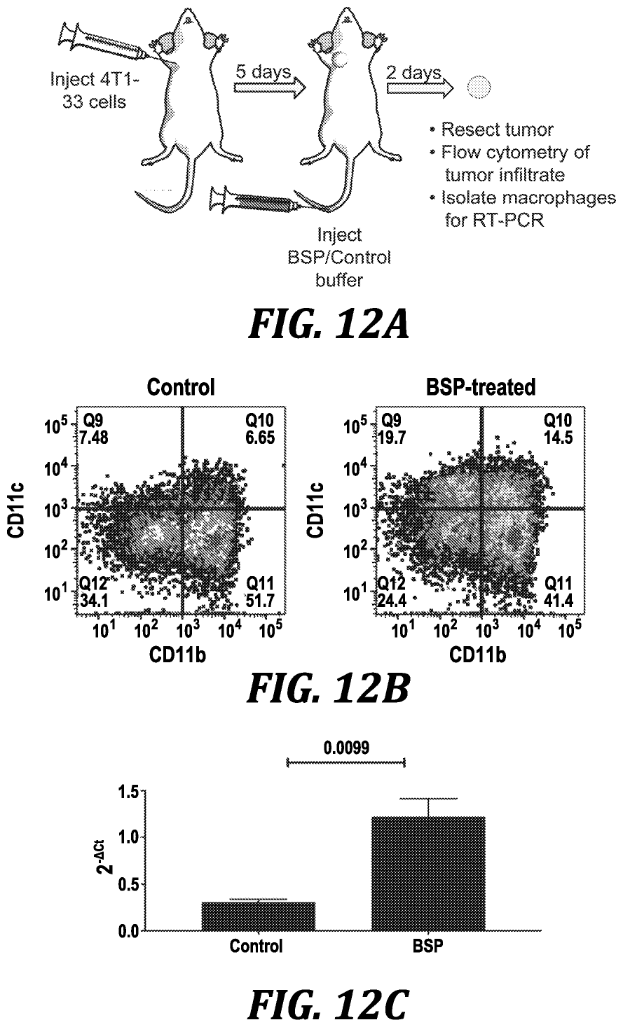

The mammary fat pads of 10 BALB/c mice were injected with parental 10⁵ 4T1s and 4T1-CD33 cells. 4T1s were injected into a top left mammary fat pad and 4T1-CD33s were injected into a top right mammary fat pad, both at 100,000-250,000 cells/injection. At five days post-cell injection, five mice were intravenously injected with 3 mgs of bi-specific protein (BSP) reagent (DLL4_{E12}-αCD33 scFv), while the remaining five mice received Hepes Buffered Saline as a control. At two days post-treatment with bi-specific protein reagent (seven days post-cell injection), tumors were individually harvested. Tumors were minced with scissors/forceps followed by enzymatic digestion using the Tumor Dissociation Kit (Miltenyi). Cells were passed through a 100 um strainer and stained with antibodies for flow cytometry. Macrophages (CD45⁺ Lin⁻ CD11b⁺ CD11c⁺ CD64⁺ CD24⁻) were sorted from bi-specific or control treated 4T1 and 4T1-CD33 tumors. See FIG. 12A. RNA was isolated from each cell population using the Nucleospin RNA XS kit (Macherey Nagel). cDNA synthesis was performed on RNA isolated from each cell population using the High Capacity RNA to cDNA kit (Applied Biosystems). Taqman PCR was used to determine the expression of Nos2 (Mm00440502_m1) relative to the housekeeping gene GusB (Mm01197698_m1), reporting (2-DCt). See FIG. 12C. As illustrated in FIG. 12C, the macrophages isolated from individuals with BSP administration had a significantly increased level of Nos2 expression, indicative of M1 status in the 4T1-CD33 tumors. This indicates monocytes have been induced via Notch signaling to develop into pro-inflammatory (M1) macrophages and/or M2 macrophages are altered via Notch signaling into an M1 phenotype.

Similar assays were conducted to analyze isolated macrophages MHCII expression. Briefly, 10⁵ 4T1-CD33 cells were injected into the flank of C57 mice. At day 5 post-cell injection, mice were intravenously injected with 3 mgs of bi-specific reagent or Hepes

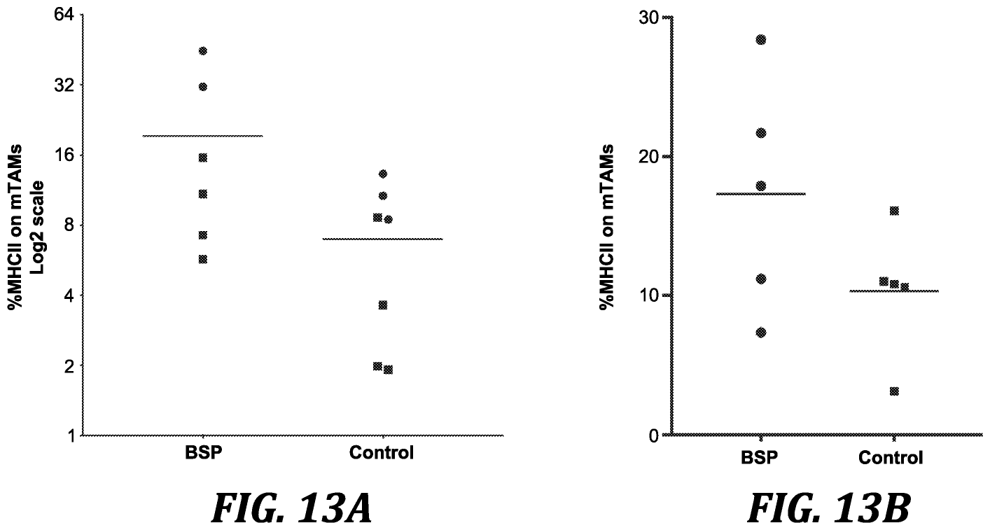

Buffered Saline as a control. At day 7 post-cell injection, 4T1 breast cancer tumors were individually resected, minced with scissors/forceps and subjected to enzymatic digestion using the Tumor Dissociation Kit (Miltenyi). Cells were passed through a 100um strainer and stained with antibodies for flow cytometry. Cells were analyzed for immune-phenotype using FACS. Breast cancer TAMs (CD45⁺ Lin^{lo} CD11b⁺ F4/80^{hi} Ly6c⁻) were analyzed for MHCII expression. As illustrated in FIG. 13B, an increase of MHCII expression was observed for the tumor associated macrophages (TAMs) upon BSP administration. This indicates that the BSP administration induces alterations of the macrophages within the tumor microenvironment. Remaining cells were analyzed for immuno-phenotype using FACS. See FIG. 12B, which indicates an increase in CD11b⁻ CD11c⁻ cells and

CD11b⁻ CD11c⁻ cells from individuals with receiving BSP treatment. Considering the role of CD11c in cellular activation of various immune cells, these results indicate that exposure of the tumor microenvironment to the bi-specific protein (BSP) agent induces immunosuppressive myeloid cells infiltrating the tumor microenvironment to exhibit a more pro-inflammatory phenotype. This may be, in part, driven by the development and recruitment of dendritic cells to the tumor microenvironment.

These data demonstrate the surprising finding that the bi-specific reagent disclosed herein and in WO 2018/017827, incorporated herein by reference in its entirety, increases Notch signaling in neighboring cells in a tumor microenvironment by trans-binding of Notch on these neighboring cells while simultaneously specifically binding to a tumor associated marker on the tumor cells. This activity can be leveraged to induce a pro-inflammatory state in the neighboring (i.e., non-tumor) cells and, thus, overcome the immune suppression typically observed in tumors. Thus, the bi-specific reagent can be implemented in overall strategies to medically intervene in solid tumor cancers.

### Example 2

The following is a description of additional investigations into the use of a bi-specific protein (BSP) reagent that binds to CD33 and to Notch receptor in vivo to result in increased Notch signaling in melanoma tumor cells. This increased signaling resulted in tumor regression, increased MHCII and/or iNOS-expressing macrophages within the tumor core, as well as increased CD3+ T cells within the tumor core.

The BSP reagent used in these investigations is described above in Example 1 and illustrated in FIG. 2. Yummer1.7 melanoma cells were transduced with lentivirus encoding human CD33 to provide a target for the BSP reagent.

Bi-specific protein (BSP) treatment reduces Yummer-CD33⁺ tumor growth.

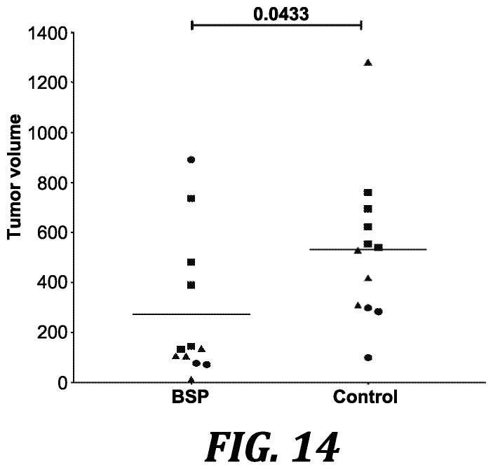

Tumor regression was observed following induction of a Notch activating field upon targeting the disclosed BSP reagent to CD33⁺ melanoma cells. 10⁶ Yummer-CD33 cells were injected into the flank of C57 mice. At days 7, 9 and 12 post-cell injection, mice were intravenously injected with 3 mgs of BSP reagent or Hepes Buffered Saline as a control. As shown in FIG. 14, at 14 days post-cell injection (experiment endpoint), reduced tumor growth was observed in mice treated with BSP as compared to control in three independent experiments.

Bi-specific targeting to Yummer-CD33⁺ cells increases the percent of MHCII-expressing tumor-associated macrophages (TAMs).

Examination of immune-phenotype of the resected tumor revealed an increase in MHCII expression among tumor associated macrophages (TAMs) following BSP reagent treatment. 10⁶ Yummer1.7-CD33 melanoma cells were injected into the flank of C57 mice. At days 7, 9 and 12 post-cell injection, mice were intravenously injected with 3 mgs of bi-specific reagent or Hepes Buffered Saline as a control. At day 14 post-cell injection, Yummer-CD33⁺ tumors were individually harvested, minced with scissors/forceps and subjected to enzymatic digestion using the Tumor Dissociation Kit (Miltenyi). Cells were passed through a 100 um strainer and stained with antibodies for flow cytometry. Cells were analyzed for immune phenotype using FACS. Melanoma TAMs (CD45⁺ Lin^{lo}) CD11b⁺ F4/80^{hi} CD169⁺ Ly6c⁻) were analyzed for MHCII expression. As shown in FIG. 13A, an increase in MHCII expression was observed among TAMs in BSP-treated mice. As MHCII is a defining feature of antigen presenting cells, this observation suggests that BSP treatment is modifying the tumor microenvironment towards an anti-tumor state.

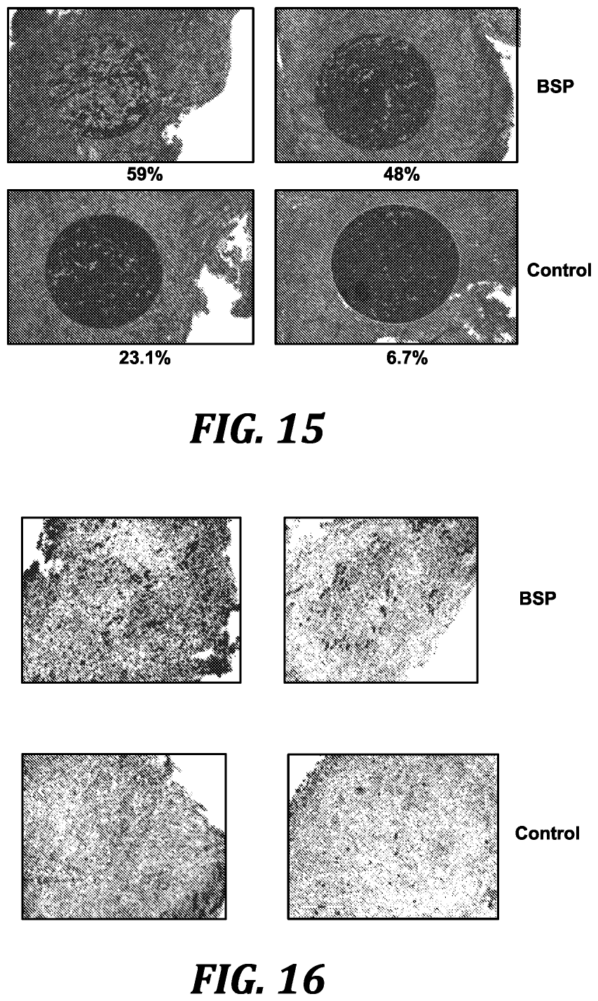

While flow cytometry has allowed the resolution of small, yet significant changes in cellular subsets following BSP treatment, immunohistochemistry allows for the assessment of BSP-induced changes in the distribution of immune cells within an intact tumor section. In initial experiments, BSP treatment was found to increase the percent of immune cells (F4/80+ MHCII+) able to infiltrate the tumor core in our murine melanoma model. Specifically, as described above, 10⁶ Yummer1.7-CD33 melanoma cells were injected into the flank of C57 mice. At days 7, 9 and 12 post-cell injection, mice were intravenously injected with 3 mgs of bi-specific reagent (BSP) or Hepes Buffered Saline (Control). At day 14 post-cell injection, tumors were resected, fixed in formalin, embedded in paraffin wax and cut into sections several microns thick. Sections were simultaneously stained with antibodies to F4/80 and MHCII and antibody binding measured using chromogenic detection. Digital images of stained slides were acquired using an Aperio ScanScope FL and analysis performed using Halo image analysis software. See FIG. 15. The number represents percent of F4/80/MHCII double positive cells among all F4/80 cells. The data are consistent with the observed increase in MHCII expression on tumor associated macrophages and tumor regression, both suggesting the bi-specific induction of an anti-tumor state.

In additional IHC experiments, BSP-treatment was found to increase the number of iNOS expressing macrophages able to infiltrate the tumor core in the murine melanoma model. Briefly, 10⁶ Yummer1.7-CD33 melanoma cells were injected into the flank of C57 mice as described above, which were then injected with BSP or Hepes Buffered Saline (control), as described above. At day 14 post-cell injection, tumors were resected, fixed in formalin, embedded in paraffin wax and cut into sections several microns thick and were stained with antibody to iNOS and antibody binding measured using chromogenic detection. Digital images of stained slides were acquired using an Aperio ScanScope FL and analysis performed using Halo image analysis software. See FIG. 16. These data are consistent with the observed increase in MHCII expression on tumor associated macrophages and tumor regression, both suggesting the BSP induction of an anti-tumor state.

These findings demonstrate that treatment with a bi-specific reagent that simultaneously binds to Notch receptor and a tumor-presented antigen switches the cellular milieu within the tumor microenvironment from an immunosuppressive to pro-inflammatory state.

While illustrative embodiments have been illustrated and described, it will be appreciated that various changes can be made therein without departing from the spirit and scope of the invention.

1. A method of inducing Notch signaling in an aggregation of cells comprising a first cell-type that expresses a cell-specific antigen and a second cell-type that expresses Notch, comprising:
contacting the aggregation of cells with a bi-specific molecule comprising a cell-targeting domain that specifically binds to the cell-specific antigen, and a Notch-binding domain that specifically binds to Notch,
wherein binding of the bi-specific molecule to the cell-specific antigen on a first cell of the first cell-type and trans-binding to Notch on a second cell of the second cell-type causes Notch signaling in the second cell.

2. The method of claim 1, wherein the first cell-type that expresses the cell-specific antigen and the second cell-type that expresses Notch are the same cell-type.

3. The method of claim 1, wherein the first cell-type that expresses the cell-specific antigen and the second cell-type that expresses Notch are different cell-types.

4. The method of claim 1, wherein the aggregation of cells is in a tumor microenvironment.

5. The method of claim 4, wherein the first cell-type comprises tumor cells and the second cell-type comprises non-tumor cells in the tumor microenvironment, wherein binding of the bi-specific molecule to the cell-specific antigen on a tumor cell and trans-binding to Notch on a non-tumor cell causes Notch signaling in the non-tumor cell.

6. The method of claim 5, wherein the non-tumor cells comprise, stromal cells, endothelial cells, and immune cells, alone or in any combination.

7. The method of claim 6, wherein the first cell-type comprises tumor cells and the second cell-type comprises immune cells, wherein binding of the bi-specific molecule to the cell-specific antigen on a tumor cell and trans-binding to Notch on an immune cell causes Notch signaling in the immune cell.

8. The method of claim 7, wherein Notch signaling in the immune cell promotes an immune-responsive state in the tumor microenvironment.

9. The method of claim 8, wherein the immune cell is a monocyte and trans-binding of the bi-specific molecule to Notch on the monocyte promotes differentiation of the monocyte into a dendritic cell.

10. The method of claim 8, wherein trans-binding of the bi-specific molecule to Notch on the immune cell promotes differentiation to M1 macrophages.

11. The method of claim 8, wherein trans-binding of the bi-specific molecule to Notch on the immune cell promotes conversion of immunosuppressive myeloid cells from an anti-inflammatory state to a pro-inflammatory state.

12. The method of claim 8, wherein trans-binding of the bi-specific molecule to Notch on the immune cell promotes an anti-tumor phenotype in a CD4⁺ T cell, in a CD8⁺ T cell, and/or in a NK cell.

13. A method of promoting a pro-inflammatory state in a tumor microenvironment comprising a tumor cell and a non-tumor cell, the method comprising:
administering to the tumor microenvironment a bi-specific molecule that comprises a cell-targeting domain that specifically binds to a cell-specific antigen expressed by the tumor cell and a Notch-binding domain that trans-binds to Notch expressed by a non-tumor cell in the tumor micro-environment, thereby inducing Notch signaling in the non-tumor cell.

14. The method of claim 13, wherein the non-tumor cell is a stromal cell, an endothelial cell, or an immune cell.

15. The method of claim 14, wherein the immune cell is a monocyte and trans-binding of the bi-specific molecule to Notch on the monocyte promotes differentiation of the monocyte into a dendritic cell.

16. The method of claim 14, wherein trans-binding of the bi-specific molecule to Notch on the immune cell promotes differentiation to an M1 macrophage.

17. The method of claim 14, wherein binding of the bi-specific molecule to Notch on the immune cell promotes conversion of immunosuppressive myeloid cells from an anti-inflammatory state to a pro-inflammatory state.

18. The method of one of claims 1-17, wherein the Notch-binding domain comprises a Notch-binding domain of a mammalian Notch receptor ligand.

19. The method of claim 18, wherein the mammalian Notch receptor ligand is a ligand to a mammalian Notch1, Notch2, Notch3, or Notch4 receptor.

20. The method of claim 18, wherein the Notch receptor ligand is a Delta protein or Jagged protein, or a derivative thereof.

21. The method of claim 20, wherein the Delta protein is Delta Like Ligand 1 (DLL1).

22. The method of claim 20, wherein the Delta protein is DLL3.

23. The method of claim 20, wherein the Delta protein is DLL4.

24. The method of claim 20, wherein the Jagged protein is Jagged 1.

25. The method of claim 20, wherein the Jagged protein is Jagged 2.

26. The method of claim 18, wherein the Notch receptor ligand is Dlk1, Dlk2, DNER, EGFL 7, and F3/contactin.

27. The method of claim 18, wherein the Notch-binding domain comprises an extracellular domain of a Delta protein or a Jagged protein, or a derivative thereof.

28. The method of claim 27, wherein the extracellular domain contains one or more mutations from wild-type resulting in enhanced affinity or specificity of the extracellular domain to the Notch receptor as compared to the wild-type extracellular domain.

29. The method of one of claim 18 or 27, wherein the Delta protein is a human Delta protein and/or wherein the Jagged protein is a human Jagged protein, or a derivative thereof.

30. The method of one of claim 18 or 27, wherein the Delta protein is a rat Delta protein and/or wherein the Jagged protein is a rat Jagged protein, or a derivative thereof.

31. The method of one of claims 1-17, wherein the Notch-binding domain comprises an antibody, an antibody-like molecule, a DARPin, an aptamer, other engineered binding modules or scaffolds, and the like, or a functional domain thereof, that binds to Notch with an affinity (K_{d}) of about 100 nM to less than 1 nM.

32. The method of one of claims 1-17, wherein the cell-targeting domain specifically binds to cell-specific antigen with an affinity (K_{d}) greater than about 100 nM.

33. The method of one of claims 1-17, wherein the cell-targeting domain comprises an antibody, an antibody-like molecule, a receptor, a DARPin, an aptamer, other engineered binding modules or scaffolds, and the like, or a functional antigen-binding domain thereof, that specifically binds to the antigen characteristic of the cell-type of interest.

34. The method of claim 31 or claim 33, wherein the antibody-like molecule is an antibody fragment and/or antibody derivative.

35. The method of claim 31 or claim 33, wherein the antibody-like molecule is a single-chain antibody, a bispecific antibody, an Fab fragment, an F(ab)₂ fragment, a V_{H}H fragment, a V_{NAR} fragment, or a nanobody.

36. The method of claim 35, wherein the single-chain antibody is a single chain variable fragment (scFv), or a single-chain Fab fragment (scFab).

37. The method of one of claims 1-17, wherein the antigen is a cell surface marker for a tumor cell.

38. The method of one of claim 5, 12-17, or 37, wherein the cancer cell or cancer progenitor cell is selected from T (leukemic) cell, breast cancer cell, prostate cell, lung cancer cell, glioblastoma, colorectal cancer cell, cervical cancer cell, melanoma cancer cell, pancreatic cancer cell, esophageal cancer cell, and the like, or a progenitor of any of the foregoing.

39. The method of claim 37, wherein the cell surface marker is CD33.

40. The method of claim 37, wherein the cell surface marker is mesothelin.

41. The method of one of claims 1-17, wherein the cell-targeting domain and the Notch-binding domain are joined by an intervening flexible linker domain.

42. The method of one of claims 1-17, wherein the molecule is a fusion polypeptide wherein the cell-targeting domain and the Notch-binding domain are polypeptides that do not naturally occur together.
